SEQUENCE LISTING (43 sequences)

SEQ ID NO: 1 - DNA, 219 bp, synthetic construct
  source 1..219: mol_type = other DNA
cagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcag

SEQ ID NO: 2 - DNA, 140 bp, synthetic construct
  source 1..140: mol_type = other DNA
ctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagc

SEQ ID NO: 3 - DNA, 109 bp, synthetic construct
  source 1..109: mol_type = other DNA
taatacaaaccccgcccgaattcgaacacgcagatgcagtcggggcggcgcggtcccaggtccacttcgcatattaaggtgacgcgtgtggcctcgaacaccgagctag

SEQ ID NO: 4 - DNA, 111 bp, synthetic construct
  source 1..111: mol_type = other DNA
taatcgtcttgtcattggcgaattcgaacacgcagatgcagtcggggcggcgcggtcccaggtccacttcgcatattaaggtgacgcgtgtggcctcgaacaccgagctag

SEQ ID NO: 5 - DNA, 90 bp, synthetic construct
  source 1..90: mol_type = other DNA
taatgaacacgcagatgcagtcggggcggcgcggtcccaggtccacttcgcatattaaggtgacgcgtgtggcctcgaacaccgagctag

SEQ ID NO: 6 - DNA, 140 bp, synthetic construct
  source 1..140: mol_type = other DNA
ctatgatgacacaaaccccgcccagcgtcttgtcattggcgaattcgaacacgcagatgcagtcggggcggcgcggtccgaggtccacttcgcatattaaggtgccggatccggcctcgaacaccgagcgaccctgcagc

SEQ ID NO: 7 - DNA, 141 bp, synthetic construct
  source 1..141: mol_type = other DNA
ctatgatgacacaaaccccgcccagcgtcttgtcattggcgaattcgaacccggatccggcagtcggggcggcgcggtccgaggtccacttcgcatattaaggtgacgcgtgtggcctcgaacaccgagcgaccctgcagc

SEQ ID NO: 8 - DNA, 140 bp, synthetic construct
  source 1..140: mol_type = other DNA
ctatgatgacacaaaccccgcccagcgtcttgtcattggcccggatccggacgcagatgcagtcggggcggcgcggtccgaggtccacttcgcatattaaggtgacgcgtgtggcctcgaacaccgagcgaccctgcagc

SEQ ID NO: 9 - DNA, 140 bp, synthetic construct
  source 1..140: mol_type = other DNA
ctatgatgacacaaaccccgcccagcgtcttgtcattggcgccggatccggcgcagatgcagtcggggcggcgcggtccgaggtccacttcgcatattaaggtgacgcgtgtggcctcgaacaccgagcgaccctgcagc

SEQ ID NO: 10 - DNA, 140 bp, synthetic construct
  source 1..140: mol_type = other DNA
ctatgatgacacaaaccccgcccagcgtcttgtcattggcgaattcgaacacgcagatgcagtcggggcggcgcggtcccggatccggttcgcatattaaggtgacgcgtgtggcctcgaacaccgagcgaccctgcagc

SEQ ID NO: 11 - DNA, 132 bp, synthetic construct
  source 1..132: mol_type = other DNA
taatacaaaccccgcccgaattcgaacacgcagatgcagtcggggcggcgcggtcccaggtccacttcgcatattaaggtgacgcgtgtggcctcgaacaccgagcgaccctgcagcgacccgcttagctag

SEQ ID NO: 12 - DNA, 134 bp, synthetic construct
  source 1..134: mol_type = other DNA
taatcgtcttgtcattggcgaattcgaacacgcagatgcagtcggggcggcgcggtcccaggtccacttcgcatattaaggtgacgcgtgtggcctcgaacaccgagcgaccctgcagcgacccgcttagctag

SEQ ID NO: 13 - DNA, 140 bp, synthetic construct
  source 1..140: mol_type = other DNA
ctatgatgacacaaaccccgcccagcgtcttgtcattggcgaattcgaacacgcagatgcagtcggggcggcgcggtccgaggtccacttcgcatattaaggtgacgcgtgtgccggatccggccgagcgaccctgcagc

SEQ ID NO: 14 - DNA, 140 bp, synthetic construct
  source 1..140: mol_type = other DNA
ctatgatgacacaaaccccgcccagcgtcttgtcattggcgaattcgaacacgcagatgcagtcggggcggcgcggtccgaggtccacttcgcatattaaggtgacgcgtgtggcctcgaacaccgccggatccggcagc

SEQ ID NO: 15 - DNA, 140 bp, synthetic construct
  source 1..140: mol_type = other DNA
cccggatccggcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagc

SEQ ID NO: 16 - DNA, 140 bp, synthetic construct
  source 1..140: mol_type = other DNA
ctatgatgacacaaaccccgcccagcgtcttgtcattggcgaattcgaacacgcagatgcagtcggggcggcgccggatccggtccacttcgcatattaaggtgacgcgtgtggcctcgaacaccgagcgaccctgcagc

SEQ ID NO: 17 - DNA, 140 bp, synthetic construct
  source 1..140: mol_type = other DNA
ctatgccggatccggccccgcccagcgtcttgtcattggcgaattcgaacacgcagatgcagtcggggcggcgcggtccgaggtccacttcgcatattaaggtgacgcgtgtggcctcgaacaccgagcgaccctgcagc

SEQ ID NO: 18 - DNA, 140 bp, synthetic construct
  source 1..140: mol_type = other DNA
ctatgatgacacaaaccccgcccagccggatccggttggcgaattcgaacacgcagatgcagtcggggcggcgcggtccgaggtccacttcgcatattaaggtgacgcgtgtggcctcgaacaccgagcgaccctgcagc

SEQ ID NO: 19 - DNA, 140 bp, synthetic construct
  source 1..140: mol_type = other DNA
ctatgatgacacaaaccccgcccagcgtcttgtcattggcgaattcgaacacgcagatgcagtcccggatccggggtccgaggtccacttcgcatattaaggtgacgcgtgtggcctcgaacaccgagcgaccctgcagc

SEQ ID NO: 20 - DNA, 140 bp, synthetic construct
  source 1..140: mol_type = other DNA
ctatgatgacacaaaccccgcccagcgtcttgtcatccggatccgggaacacgcagatgcagtcggggcggcgcggtccgaggtccacttcgcatattaaggtgacgcgtgtggcctcgaacaccgagcgaccctgcagc

SEQ ID NO: 21 - DNA, 140 bp, synthetic construct
  source 1..140: mol_type = other DNA
ctatgatgacacaaaccccgcccagcgtcttgtcattggcgaattcgaacacgcagatgcaccggatccggcgcggtccgaggtccacttcgcatattaaggtgacgcgtgtggcctcgaacaccgagcgaccctgcagc

SEQ ID NO: 22 - DNA, 141 bp, synthetic construct
  source 1..141: mol_type = other DNA
ctatgatgacacaaaccccgcccagcgtcttgtcattggcgaattcgaacacgcagatgcagtcggggcggcgcggtccgaggtccacttcgcatattaccggatccgggtgtggcctcgaacaccgagcgaccctgcagc

SEQ ID NO: 23 - DNA, 139 bp, synthetic construct
  source 1..139: mol_type = other DNA
ctatgatgacacaaaccccgcccagcgtcttgtcattggcgaattcgaacacgcagatgcagtcggggcggcgcggtccgaggtccacttcccggatccggtgacgcgtgtggcctcgaacaccgagcgaccctgcagc

SEQ ID NO: 24 - DNA, 140 bp, synthetic construct
  source 1..140: mol_type = other DNA
ctatgatgaccggatccgggcccagcgtcttgtcattggcgaattcgaacacgcagatgcagtcggggcggcgcggtccgaggtccacttcgcatattaaggtgacgcgtgtggcctcgaacaccgagcgaccctgcagc

SEQ ID NO: 25 - DNA, 140 bp, synthetic construct
  source 1..140: mol_type = other DNA
ctatgatgacacaaaccggatccggcgtcttgtcattggcgaattcgaacacgcagatgcagtcggggcggcgcggtccgaggtccacttcgcatattaaggtgacgcgtgtggcctcgaacaccgagcgaccctgcagc

SEQ ID NO: 26 - DNA, 1125 bp, Cricetulus griseus
  source 1..1125: mol_type = other DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagacaaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaggactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcacaggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagatcgccgcccctctgccaattgtgacccctttgcagtgacagaagccatcgtccgcacatgccttctcaatgagactggcgacgagcccttccaatacaaaaactaatga

SEQ ID NO: 27 - DNA, 122 bp, Simian virus 40
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 28 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 29 - DNA, 56 bp, synthetic construct
  source 1..56: mol_type = other DNA
gtcgacgccgccaccatgagagacgtctaaggtaacgtctcgtaatgagcggccgc

SEQ ID NO: 30 - DNA, 55 bp, synthetic construct
  source 1..55: mol_type = other DNA
tctagactaagccgccaccagagacgtactaagttacgtctctcgatgagaattc

SEQ ID NO: 31 - DNA, 1119 bp, Homo sapiens
  source 1..1119: mol_type = other DNA
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctccagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaat

SEQ ID NO: 32 - DNA, 1126 bp, Mus musculus
  source 1..1126: mol_type = other DNA
atggccacctcagcaagttcccacttgaacaaaggcatcaagcaaatgtacatgtccctgccccagggtgagaaagtccaagccatgtatatctgggttgatggtaccggagaaggactgcgctgcaagacccgtaccctggactgtgagcccaagtgtgtggaagagttacctgagtggaactttgatggctctagtacctttcagtctgaaggctccaacagcgacatgtacctccatcctgttgccatgtttcgagaccccttccgcagagaccccaacaagctggtgctatgtgaagttttcaagtataaccggaaacctgcagagaccaacttgaggcacatctgtaaacggataatggacatggtgagcaaccagcacccctggtttggaatggagcaggaatatactcttatgggaacagacggccacccatttggttggccttccaatggcttccctggaccccaaggcccgtattactgcggtgtgggagcagacaaggcctacggcagggacatcgtggaggctcactaccgggcctgcttgtatgctggagtcaagattacggggacaaatgcggaggttatgcctgcccagtgggaattccaaataggaccctgtgaggggatccgaatgggagatcatctttggatagcccgttttatcttgcatcgggtgtgcgaagactttggggtgatagcaacctttgaccccaagcccattccagggaactggaatgttgcaggctgccataccaacttcagcaccaaggccatgcgggaggagaatggtctgaagtgcattgaggaggccattgacaaactgagcaagaggcaccagtaccacattcgcgcctacgatcccaaggggggcctggacaatgcccgtgctctgactggattccacgaaacctccaacatcaacgacttttctgctggtgttgccaaccgcggtgccagtatccgcattccccggactgtcggccaggagaagaagggctactttgaagaccgtcgccttcgtgccaattgtgacccctatgcggtgacagaagccatcgtccgcacgtgtctcctcaacgaaacaggcgacgaacccttccaatacaagaactaagtga

SEQ ID NO: 33 - DNA, 1122 bp, Onychomys torridus
  source 1..1122: mol_type = other DNA
atggccacctcagcaagttcccacttgaacaaaggcatcaagcaaatgtacatgtccctgccccagggtgagaaagtgcaagccatgtatatctgggtggacggtaccggagaaggactgcgttgtaagacccgcaccctggactgtgagcccaagtgtgtcgaagagttacctgagtggaattttgatggatctagtacctttcagtccgagggctccaacagtgacatgtatctcagccctgttgccatgtttcgggaccccttccgcaaagagcccaacaagttggtgttctgtgaagttttcaagtacaaccggaagcctgcagagaccaatttaagacacacctgtaaacggataatggacatggtgagcagccagcacccctggtttggaatggaacaggaatacactctcatgggaacagatgggcacccttttggttggccatccaatggcttccctgggccccaaggtccatattactgtggcgtgggagcagacaaagcctatggcagggatattgtggaggcccactaccgggcctgcttgtatgctggagtcaagattacaggaacaaatgctgaggtcatgcctgcccagtgggaattccagataggaccgtgtgaaggaatccgcatgggagatcatctctgggtggcccggttcatcttgcatcgcgtatgtgaagactttggggtgatagcaacctttgaccccaagcccattcctgggaactggaatggcgcaggctgccataccaactttagcaccaaggccatgcgggaggagaacggtctgaagtacattgaggaggccattgagaaactgagcaagcggcaccagtaccacattcgcgcctacgatcccaaggggggcctggacaacgcccggcgtctgactggattccacgaaacctccaacatcaacgacttttctgccggcgtggccaaccgcagcgccagcatccgcattccccggactgtcggccaggagaagaggggttacttcgaagaccgtcgtccttctgccaactgtgacccgtttgccgtgacagaagccatcgtccgcacatgccttctcaacgagactggcgacgagcccttccagtacaagaactaa

SEQ ID NO: 34 - protein, 340 aa, synthetic construct
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 35 - protein, 341 aa, synthetic construct
MVRGKPISKEIRVLIRDYFKSGKTLTEISKQLNLPKSSVHGVIQIFKKNGNIENNIANRGRTSAITPRDKRQLAKIVKADRRQSLRNLASKWSQTIGKTVKREWTRQQLKSIGYGFYKAKEKPLLTLRQKKKRLQWARERMSWTQRQWDTIIFSDEAKFDVSVGDTRKRVIRKRSETYHKDCLKRTTKFPASTMVWGCMSAKGLGKLHFIEGTVNAEKYINILQDSLLPSIPKLSDCGEFTFQQDGASSHTAKRTKNWLQYNQMEVLDWPSNSPDLSPIENIWWLMKNQLRNEPQRNISDLKIKLQEMWDSISQEHCKNLLSSMPKRVKCVMQAKGDVTQF

SEQ ID NO: 36 - protein, 343 aa, synthetic construct
MEMMLDKKQIRAIFLFEFKMGRKAAETTRNINNAFGPGTANERTVQWWFKKFRKGDESLEDEERSGRPSEVDNDQLRAIIEADPLTTTREVAEELNVDHSTVVRHLKQIGKVKKLDKWVPHELSENQKNRRFEVSSSLLLRNNNEPFLDRIVTCDEKWILYDNRRRSAQWLDREEAPKHFPKPNLHQKKVMVTVWWSAAGVIHYSFLNPGETITSEKYCQQIDEMHRKLQRLQPALVNRKGPILLHDNARPHVAQPTLQKLNELGYEVLPHPPYSPDLSPTDYHFFKHLDNFLQGKRFHNQQDAENAFQEFVESRSTDFYATGINKLISRWQKCVDCNGSYFD

SEQ ID NO: 37 - protein, 402 aa, synthetic construct
MIHTNLKKKFSYFILAFLLFALICVWKKGSYEALKLQAKEFQVTRSLEKLAMRSGSQSMSSSSKQDPKQDSQVLSHARVTAKVKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 38 - protein, 529 aa, synthetic construct
MKPHLKQWRQRMLFGIFAWGLLFLLIFIYFTDSNPAEPVPSSLSFLETRRLLPVQGKQRAIMGAAHEPSPPGGLDARQALPRAHPAGSFHAGPGDLQKWAQSQDGFEHKEFFSSQVGRKSQSAFYPEDDDYFFAAGQPGWHSHTQGTLGFPSPGEPGPREGAFPAAQVQRRRVKKRHRRQRRSHVLEEGDDGDRLYSSMSRAFLYRLWKGNVSSKMLNPRLQKAMKDYLTANKHGVRFRGKREAGLSRAQLLCQLRSRARVRTLDGTEAPFSALGWRRLVPAVPLSQLHPRGLRSCAVVMSAGAILNSSLGEEIDSHDAVLRFNSAPTRGYEKDVGNKTTIRIINSQILTNPSHHFIDSSLYKDVILVAWDPAPYSANLNLWYKKPDYNLFTPYIQHRQRNPNQPFYILHPKFIWQLWDIIQENTKEKIQPNPPSSGFIGILIMMSMCREVHVYEYIPSVRQTELCHYHELYYDAACTLGAYHPLLYEKLLVQRLNMGTQGDLHRKGKVVLPGFQAVHCPAPSPVIPHS

SEQ ID NO: 39 - DNA, 71 bp, synthetic construct
  source 1..71: mol_type = other DNA
gtcgacgcatacggaaatgcatattcatcgtatcgcgatattgtgtctatcgatattcaccgagcggccgc

SEQ ID NO: 40 - DNA, 87 bp, synthetic construct
  source 1..87: mol_type = other DNA
tctagactattccgctgatcattgcttacacacgtgcggtacgtcgggcgcgcctgggcatacggaaccccgggacgtctctcgata

SEQ ID NO: 41 - protein, 406 aa, synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 42 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 43 - DNA, 71 bp, synthetic construct
  source 1..71: mol_type = other DNA
gtcgacgcatacggaacctaggattcatcgtatcgcgatattgtgtctatcgatattcaccgagcggccgc

(19) United States
(12) Patent Application Publication
(10) Pub. No.: US 2025/0304997 A1
(43) Pub. Date: Oct. 2, 2025

(54) POLYNUCLEOTIDES WITH SELECTION MARKERS

(71) Applicant: Revvity Discovery Limited, Waterbeach (GB)

(72) Inventors: Delphine Cougot, Cambridge (GB); Ana Rebocho, Cambridge (GB); Tanaya Surve, Cambridge (GB); Tulshi Patel, Cambridge (GB); James Fleming, Cambridge (GB); Mario Pereira, Cambridge (GB); Amanda Smith, Cambridge (GB); Karen Sandeman, Ayr Ayrshire (GB)

(21) Appl. No.: 18/836,796

(22) PCT Filed: Feb. 9, 2023
(86) PCT No.: PCT/GB2023/050293

§ 371 Date: Aug. 8, 2024

(30) Foreign Application Priority Data
Feb. 9, 2022 (GB) 2201662.0
Jun. 14, 2022 (GB) 2208714.2

(51) Int. Cl. C12N 15/86; C07K 16/32; C12N 9/00

(52) U.S. Cl. CPC C12N 15/86; C07K 16/32; C12N 9/93; C12Y 603/01002; C07K 2317/24; C12N 2740/15043; C12N 2800/90

(57) ABSTRACT

A polynucleotide with a selection marker sequence under the control of a desirable HSVMin promoter leads to improved efficiency for biotechnology applications. The polynucleotide may, for example, be a transposon for use with Tc1/mariner systems. Through various embodiments of the present disclosure, one is able to efficiently and effectively introduce cargo regions from polynucleotides such as those carrying a glutamine synthetase gene into a host's DNA.

### CROSS REFERENCE TO RELATED APPLICATIONS

This application is a U.S. national stage of International Patent Application No. PCT/GB2023/050293, filed Feb. 9, 2023, which claims priority from GB Patent Application No. 2208714.2, filed Jun. 14, 2022, and GB Patent Application No. 2201662.0, filed Feb. 9, 2022. The entire contents of these applications are incorporated herein by reference in their entirety.

### INCORPORATION BY REFERENCE OF MATERIAL SUBMITTED ELECTRONICALLY

A Sequence Listing, which is a part of the present disclosure, is submitted concurrently with the specification as an electronic file. The name of the electronic file containing the Sequence Listing is “P36347WO1 Sequence Listing v2.xml.” The Sequence Listing was created on Feb. 7, 2023, and is 49,331 bytes in size. The subject matter of the Sequence Listing is incorporated by reference herein in its entirety.

### FIELD

The present disclosure relates to the field of selection markers.

### BACKGROUND

Selection markers are valuable tools in the biotechnology industry. They allow researchers to introduce genetic sequences of interest into a desired cell host and to cause the host cell to maintain expression of those genetic sequences, often by promoting their integration into the cells' chromosomal DNA. Selection markers may complement metabolic or enzymatic activities that are absent in the host cell but that are essential for the survival of the cell under certain culture conditions. Such selection markers may include, for example, critical enzymes required for the metabolism of essential nutrients (such as glutamine synthetase or dihydrofolate reductase amongst others) or may provide an antibiotic resistant enzyme (neomycin phosphotransferase gene). Selection markers are usually incorporated into an expression vector where a given gene of interest will also be introduced. After the selection markers and gene of interest are introduced, cells are placed under selection conditions in which they will not be able to survive in the absence of such selection marker function (for example, by starving the culture of an essential nutrient or placing the cells under conditions in which an antibiotic is present). Under such selection conditions, cells that have incorporated the expression vector containing the selection marker will be able to survive, whereas those that have not, will die or fail to proliferate. These expression vectors, containing selection markers and genes of interest can be varied in nature, from plasmid DNA vectors to viral vectors or transposons.

In the case of plasmid DNA expression vectors, DNA is typically linearized and then transfected into the expression host by means of electroporation or lipids, and the DNA either is incorporated as episomal DNA or integrates randomly in one or several positions of the chromosome in the genome of the host cell. Those cells that successfully incorporate the expression vector or integrate its DNA into their chromosomes can survive under selection conditions and will also successfully express the genes of interest that are incorporated into the expression vector DNA.

Similarly, viral vectors incorporating genes encoding for a given selection marker, as well as one or several genes of interest can be used to transduce host cells and promote the integration of their genetic material into the host cell genome. In the case of retroviruses, the genetic material (RNA) is first transcribed into DNA that then is actively integrated into the host cell genome.

Another strategy for introducing selection markers into cells makes use of transposons, which are mobile polynucleotide sequences with the ability to move from one position within a given genome to another position within the genome or from a DNA vector to a host genome. Over the past few decades, the biotechnology industry has used transposons for both gene delivery and mutagenesis in many different cell types and organisms including, but not limited to rodents, avian cells, non-human primates or humans.

Both RNA and DNA transposons exist, but DNA transposons, particularly members of the Tc1/mariner family, have received a great deal of focus from researchers. One reason for this focus is that researchers have estimated that the human genome contains thousands of Tc1/mariner type transposons, but no endogenous genes for any corresponding transposases, which are the enzymes necessary to cut and paste the transposons. Thus, Tc1/mariner transposons present a significant opportunity for working with the human genome.

In the late 1990s, researchers reconstructed a functional Tel-like transposon system from sequences found in salmonid fish by creating a synthetic transposase named Sleeping Beauty. In the wake of the creation of the Sleeping Beauty system, researchers designed different vectors that contained polynucleotide sequences that the Sleeping Beauty transposase could mobilize, i.e., vectors from which polynucleotide sequences of interest could be cut and inserted into a host genome. The genetic material that is cut and inserted is located between the ITRs, (inverted terminal repeats) and may be referred to as the “cargo” of the transposon vector.

When using a transposon system, researchers look to optimize at least two parameters: (1) integration efficiency, which refers to the average number of copies of the cargo from transposons that are integrated into each cell; and (2) expression efficiency, which refers to the level of expression of a given gene incorporated in the cargo when incorporated into the cell genome. Although researchers have worked with Tc1/mariner family systems for more than two decades, these systems have yet to be optimized for all applications. Various embodiments of the present disclosure are directed to optimizing one or both of the aforementioned parameters as well as the efficient use of other transposons and other vectors for the introduction of selection markers and other polynucleotide sequences into DNA.

### SUMMARY

The present disclosure provides novel and non-obvious improvements to selection marker systems in which selection cassettes and expression cassettes incorporated in an expression vector are introduced into cells and host DNA. When using certain promoters in these selection marker systems, one sees an improvement in one or both of the number of integrated copies and expression efficiency.

According to a first embodiment, the present disclosure provides a polynucleotide comprising a cargo region, wherein the cargo region comprises: (a) an expression cassette; and (b) a selection cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides.

According to another embodiment, the present disclosure provides a polynucleotide comprising a selection cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides.

According to another embodiment, the present disclosure provides a polynucleotide comprising one or more expression cassettes and a selection cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides.

According to a second embodiment, the present disclosure provides a polynucleotide comprising a cargo region, wherein the cargo region comprises: (a) a first expression cassette, (b) a second expression cassette; and (c) a selection cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides.

According to a third embodiment, the present disclosure provides a polynucleotide comprising a cargo region, wherein the cargo region comprises: (a) a first expression cassette; (b) a second expression cassette; (c) a selection cassette; and (d) a third expression cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence complementary to SEQ ID NO: 14 or SEQ ID NO: 24 over a span of at least 100 nucleotides and the third expression cassette comprises a polynucleotide sequence that codes a polypeptide that is capable of causing post-translation modification of one or both of a polypeptide coded by a polynucleotide sequence within the first expression cassette and a polypeptide coded by a polynucleotide sequence within the second expression cassette.

According to a fourth embodiment, the present disclosure provides a transposon vector comprising a cargo region, wherein the cargo region comprises: (a) a first expression cassette, wherein the first expression cassette is oriented in a first direction, (b) a second expression cassette, wherein the second expression cassette is oriented in the first direction; and (c) a selection cassette, wherein the selection cassette is located between the first expression cassette and the second expression cassette and the selection cassette is oriented in a second direction, wherein the first direction and the second direction are opposite directions, and further wherein the selection cassette comprises: (i) an HSVMin promoter sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides, and (ii) a selection marker, e.g., glutamine synthetase sequence, wherein the glutamine synthetase sequence codes for a glutamine synthetase protein, wherein the cargo region is capable of being cut from the transposon vector by a Tc1/mariner transposase protein. Optionally, the transposon vector comprises a third expression cassette, wherein the third expression cassette comprises a polynucleotide sequence that codes a polypeptide that is capable of causing post-translation modification of one or both of a polypeptide coded by a polynucleotide sequence within the first expression cassette and a polypeptide coded by a polynucleotide sequence within the second expression cassette.

According to a fifth embodiment, the present disclosure provides a genetic delivery system comprising a plasmid DNA vector disclosed herein. In some embodiments, the plasmid DNA vector is a random integration vector.

According to a sixth embodiment, the present disclosure provides a genetic delivery system comprising a transposon vector disclosed herein and either (1) a transposase protein, or (2) a DNA plasmid or an mRNA wherein the DNA plasmid or the mRNA encode a transposase protein. The transposase protein has the ability to catalyze translocation of the transposon from the vector into host DNA.

According to a seventh embodiment, the present disclosure provides a method for introducing a nucleotide sequence into host DNA. The method comprises exposing the host DNA to a polynucleotide e.g., a plasmid DNA vector or a transposon vector disclosed herein. The host DNA may, for example, be located in a cell and be chromosomal DNA or extra-chromosomal DNA.

According to an eighth embodiment, the present disclosure provides another method for introducing a polynucleotide sequence into host DNA. The method comprises exposing the host DNA to a genetic delivery system disclosed herein. The genetic delivery system may comprise any of the polynucleotides disclosed herein and optionally also comprise one more proteins or other polynucleotides that code for proteins that facilitate translocation or integration of a cargo region into the host DNA.

The host DNA may, for example, be located in a cell and be chromosomal DNA or extra-chromosomal DNA.

According to a ninth embodiment, the present disclosure provides a modified cell, wherein the modified cell comprises both host DNA and either a polynucleotide, e.g., a transposon, or a genetic delivery system of the present disclosure.

According to a tenth embodiment, the present disclosure provides a modified cell, wherein the modified cell comprises host DNA, wherein within the host DNA is a cargo region. The cargo region may, for example, comprise: (a) one or several expression cassettes; and (b) a selection cassette. When there is a plurality of expression cassettes, the selection cassette may be located downstream or upstream of all of the expression cassettes, or the selection cassette may be located between a pair of consecutive expression cassettes. In some embodiments, each expression cassette is oriented in a first direction, and the selection cassette is oriented in a second direction, wherein the first direction and the second direction are opposite directions. The selection cassette may comprise: (i) an HSVMin promoter sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides, and (ii) a selection marker sequence, e.g., a glutamine synthetase sequence, wherein the glutamine synthetase sequence codes for a glutamine synthetase protein.

According to an eleventh embodiment, the present disclosure provides a use of a modified cell of the present disclosure to generate a biologic material such as a protein, including but not limited to monoclonal antibodies, Fc-Fusion, bi-specific or multi-specific or any other biopharmaceuticals and difficult to express proteins.

According to a twelfth embodiment, the present disclosure provides a use of a modified cell of the present disclosure to develop a biopharmaceutical or a non-protein biotherapeutic product such as viral vectors, non-coding RNAs such as miRNA, shRNA, or long non-coding RNA.

According to a thirteenth embodiment, the present disclosure provides a bioproduction method for producing a product. The method comprises utilizing a modified cell of any of the embodiments of the present disclosure to produce the desired product. The method may, for example, comprise bioreactor cultures utilizing fed-batch processes, perfusion feeding, intensified or continuous manufacturing processes.

According to a fourteenth embodiment, the present disclosure provides a method for integrating an exogenous nucleotide sequence comprising a cargo region into a nucleotide sequence of a host cell. The method comprises introducing one or more polynucleotides of the present disclosure into a cell or exposing one or more polynucleotides of the present disclosure to a host cell's DNA.

According to a fifteenth embodiment, the present disclosure provides a first polynucleotide and a second polynucleotide. The first polynucleotide comprises: (a) a first expression cassette; (b) a second expression cassette; and (c) a selection cassette, wherein the selection cassette comprises: an HSVMin promoter sequence and a selection marker sequence, wherein the HSVMin promoter sequence is from 120 to 160 nucleotides long and comprises at least 80% sequence identity to SEQ ID NO: 14 or SEQ ID NO: 24 or comprises at least 80% sequence identity to a sequence that is complementary to SEQ ID NO: 14 or SEQ ID NO: 24 over a span of at least 100 nucleotides. The second polynucleotide comprises a third expression cassette and optionally, a selection marker such as an antibiotic selection marker. The selection marker that is coded by the second polynucleotide may be different from or the same as a selection marker that is coded by a sequence within the selection cassette. The third expression cassette may comprise a polynucleotide sequence that codes a polypeptide that is capable of causing post-translation modification of one or both of a polypeptide coded by a polynucleotide sequence within the first expression cassette and a polypeptide coded by a polynucleotide sequence within the second expression cassette.

In this embodiment, the first polynucleotide and the second polynucleotide are separate polynucleotides and each or both may be or be part of a transposon vector or other type of vector. The first polynucleotide and the second polynucleotide may be introduced simultaneously or sequentially into a cell to form a modified cell. These cells may be used in the same applications as other modified cells described herein.

When determining the effectiveness of a polynucleotide that codes for a selection marker sequence to integrate into the host DNA, in many applications two parameters are particularly important: gene copy number (also referred to as integration efficiency) and productivity (also referred to as expression efficiency). Gene copy number refers to the number of copies integrated into the host cell genome, and productivity refers to the amount of protein of interest secreted by the cells. Through the various embodiments disclosed herein, one may efficiently and effectively deliver genetic material to host DNA and/or express proteins from the host DNA.

### BRIEF DESCRIPTION OF THE FIGURES

FIG. 1 is a growth profile graph that provides a representation of the viable cell density (VCD) of CHOSOURCE™ glutamine synthetase (GS) knock-out (KO) cells (from Horizon Discovery™, catalog number HD-BIOP3, herein referred to as GS KO cells or GS KO cell line) transfected with vectors comprising GB14 (SEQ ID NO: 14) and GB24 (SEQ ID NO: 24) promoters, during selection in glutamine free media, as compared to WT, wild-type promoter (SEQ ID NO: 1) and a non-transfected control.

FIG. 2 is a cell viability graph that provides a representation of the viability of GS KO cells transfected with vectors comprising GB14 (SEQ ID NO: 14) and GB24 (SEQ ID NO: 24) promoters, during selection in glutamine free media, as compared to WT (SEQ ID NO: 1) and a non-transfected control.

FIG. 3 is a graph that provides a representation of the number of GS gene copies integrated into the host cell DNA. The copy number was determined using droplet digital PCR (ddPCR) for WT promoter (SEQ ID NO: 1), GB14 promoter (SEQ ID NO: 14), and GB24 promoter (SEQ ID NO: 24) within the selection cassette.

FIG. 4 is a pool productivity assessment graph that provides a representation of the titer obtained for cells transfected with vectors containing GB14 (SEQ ID NO: 14) and GB24 (SEQ ID NO: 24) promoters, as compared to the WT promoter (SEQ ID NO: 1).

FIG. 5 is a graph that compares the productivity of twenty-four different promoters when used in transposon vectors, as compared to the WT promoter in the selection cassette.

FIG. 6 is an example of a linear schematic of the gene of interest (cargo) region located between ITRs of a transposon vector encompassed by the present disclosure. Element 100 represents the gene of interest (“GOI”) or cargo region, located between a first ITR 101 and a second ITR 102. Within the GOI region, there are a first expression cassette 120, a second expression cassette 130, and a selection cassette 140. Elements 121, 122 and 123 correspond to an EF-1α promoter, a heavy chain antibody coding region (“HC”) and bGH pA, respectively. Elements 131, 132 and 133 correspond to an EF-1α promoter, a light chain antibody coding region (“LC”) and SV40 pA, respectively. Elements 141, 142 and 143 correspond to HSVMin promoter, glutamine synthetase (“GS”) coding sequence and SV40 pA, respectively.

FIG. 7 is another example of a linear schematic of a cargo region located between tandem repeats of a transposon vector encompassed by the present disclosure. Element 180 corresponds to the post-translational modification cassette. Within element 180, elements 181 and 182 correspond to the CMV promoter sequence and the sialyltransferase (ST6) sequence, respectively. Elements 191 and 192 correspond to the first ITR and second ITR, labeled as LIR and RIR, respectively.

FIG. 8 is another example of a linear schematic of a cargo region located between tandem repeats of a transposon vector encompassed by the present disclosure.

FIG. 9. FIG. 9(A) is an example of a linear schematic of a cargo region located between tandem repeats that may be used in combination with a separate vector that comprises the elements of the linear schematic of a polynucleotide as shown in FIG. 9B, which comprises a third expression cassette. FIG. 9(B) shows its LIR, 910, a selection marker e.g., neomycin 920, a fourth promoter e.g., EF-1α 930, a fifth promoter, 940, and a polynucleotide sequence that codes for a post-translation protein 950 followed by the RIR 960.

FIG. 10. FIG. 10(A) is a growth profile graph that provides a representation of the viable cell density of pools from GS KO cells and CHOSOURCE™ ADCC+ cells (from Horizon Discovery™, catalog number HD-BIOP004, herein referred to as ADCC+ cells or ADCC+ cell line), during selection in glutamine free media. The two different cell line hosts were transfected with vectors comprising GB24 (SEQ ID NO: 24) promoter as compared to WT wild-type promoter (SEQ ID NO: 1) and a non-transfected control for both cell types. FIG. 10(B) is a graph representing the percentage viability for pools of GS KO and ADCC+ cell line hosts transfected with vectors comprising GB24 (SEQ ID NO: 24) promoter, during selection in glutamine free media, as compared to WT, wild-type promoter (SEQ ID NO: 1) and a non-transfected control for both cell line hosts. Each point on the graph represents the average of 3 pools for WT and GB24 and one pool of untransfected control. Error bar represents standard deviation.

FIG. 11 is a graph that provides a representation of the GS gene copy number integrated into the different cell line hosts in glutamine free media. The copy number was determined using ddPCR for WT promoter (SEQ ID NO: 1) and GB24 promoter (SEQ ID NO: 24) within the selection cassette in GS KO and ADCC+ cell line hosts. The graph represents the Glutamine Synthase (GS) copy number variation as observed in the pools recovered from selection. The assay was performed in triplicate. Error bars represent standard deviation.

FIG. 12. FIG. 12(A) is a growth profile graph that provides a representation of the viable cell density of the 14-day fed-batch overgrowth culture of pools from GS KO and ADCC+ cell line hosts transfected with vectors comprising GB24 promoter (SEQ ID NO: 24), during selection in glutamine free media, as compared to the wild-type promoter WT (SEQ ID NO: 1). FIG. 12(B) represents the percentage viability of the pools during the 14-day fed-batch overgrowth experiment. Each point on the graph represents the average of 3 pools for WT and GB24 in GS KO and ADCC+ cell lines. Error bar represents standard deviation.

FIG. 13 is a productivity profile graph from fed-batch overgrowth culture that provides a representation of the titer obtained for GS KO and ADCC+ cell lines transfected with vectors containing GB24 promoter (SEQ ID NO: 24), as compared to the WT promoter (SEQ ID NO: 1). Each bar represents the average of three expressing pools generated for each construct. The samples were analysed in duplicate and error bars represent standard deviation. Results are expressed relative to the WT promoter.

FIG. 14 is a graph representing the expression (mRNA levels) of a post-translational modification enzyme (ST6Gal1) and of the enzyme ST6Gal1 and trastuzumab (Ttz) antibody expressed together. Each bar represents the average of three expressing pools generated for each construct. The ST6Gal1 mRNA levels were detected by RT-qPCR in non-transfected GS KO cells (“GS KO” in the figure) and GS KO cells transfected with either ST6Gal1 (“ST6Gal1” in the figure) or ST6Gal1 and Ttz (“ST6Gal1 & Ttz” in the figure).

FIG. 15 is a histogram showing a flow cytometry assay for the analysis of GS KO pools expressing green fluorescent protein (GFP). 10,000 cells were analysed per pool, live single cells were isolated using SSC-A/FSC-A and FSC-H/FSC-A gates. The histogram depicts GFP expression of four transfected pools over the non-transfected GS KO cell line using a FL2-A:EGFP-A filter and laser. The table depicts the flow cytometry data, i.e., the frequency of GFP expressing cells (pools 1-4) and non-GFP expressing cells (GS KO) within transfected and non-transfected pools, respectively.

| | Sample | Freq. of | Subset Name |
|---|---|---|---|
| | Name | Parent | (indented) |
| [symbol] | GS KO_Data Source - 1.fcs | 0 | EGFP-A+ |
| [symbol] | GS KO_Data Source - 1.fcs | 100 | EGFP-A− |
| [symbol] | Pool 1_Data Source - 1.fcs | 82.5 | EGFP-A+ |
| [symbol] | Pool 1_Data Source - 1.fcs | 17.5 | EGFR-A− |
| [symbol] | Pool 2_Data Source - 1.fcs | 82.4 | EGFP-A+ |
| [symbol] | Pool 2_Data Source - 1.fcs | 17.6 | EGFP-A− |
| [symbol] | Pool 3_Data Source - 1.fcs | 75.4 | EGFP-A+ |
| [symbol] | Pool 3_Data Source - 1.fcs | 24.6 | EGFP-A− |
| [symbol] | Pool 4_Data Source - 1.fcs | 83.1 | EGFP-A+ |
| [symbol] | Pool 4_Data Source - 1.fcs | 16.9 | EGFP-A− |

FIG. 16 is a graph that compares the GS copy number of twenty-four different promoters when used in transposon vectors, as compared to the WT promoter in the selection cassette. The graph represents the number of GS copies integrated into the cell line host DNA of GS KO pools generated with transposon vectors comprising the different HSV promoter sequences. Each transposon vector was used to generate three pools. The GS gene copy number was determined using ddPCR. GS specific primers and probes were designed for the ddPCR and β2-Microbulin was used as a reference gene. Assay was performed in triplicate and error bars represent standard deviation.

FIG. 17. FIG. 17(A) is a graph that shows the genetic stability of the integration in clones over 90 generations, determined using the GS copy-number. The graph represents the GS copy number variance of GB14 and GB24 clones. For each clone, the copy number was assessed at generation 0 (Gen 0) and at generation 90 (Gen 90) and data is represented normalised to Gen 0. FIG. 17(B) is a graph that shows the clone productivity profile for the GB14 and GB24 clones in a fed-batch overgrowth experiment. For each clone, productivity was assessed at generation 0 (Gen 0) and at generation 90 (Gen 90) and data is represented normalised to Gen 0. Clones are considered stable when titre variation between Gen 0 and Gen 90 is below 30%. The titer was analysed on day 14 at harvest using Octet® Protein A Biosensor.

FIG. 18 is a productivity profile graph from fed-batch overgrowth culture that provides a representation of the titer obtained for GS KO pools transfected with vectors comprising (i) GB24 promoter (SEQ ID NO: 24) and Ttz (in the figure “Ttz”) and (ii) GB24 promoter (SEQ ID NO: 24) and Etanercept (Etn, in the figure “Etn”). Each bar represents the average of three expressing pools generated for Etn and one pool for Ttz. The samples were analysed in duplicate and error bars represent standard deviation. Results are expressed relative to Ttz.FIG. 19. FIG. 19(A) is a graph representing overlayed electropherograms of glycan standards, showing the mono-sialylated peak (peak 1) at the bottom and di-sialylated peak (peak 2) at the top. The lower marker is an internal standard used in each run to align data from each sample well. FIG. 19(B) depicts overlayed electropherograms of sialylated Ttz (co-expression of Ttz under the control of GB24 and ST6Gal1; top) and non-sialylated Ttz (expression of Ttz; bottom). The sialylated structures detected are identified and labelled on the figure. These structures were identified based on an alignment with the glycan standards, shown in FIG. 19A.

### DETAILED DESCRIPTION

Reference will now be made in detail to various embodiments of the present disclosure, examples of which are illustrated in the accompanying Figures. In the following description, numerous specific details are set forth in order to provide a thorough understanding of the present disclosure. However, unless otherwise indicated or implicit from context, the details are intended to be examples and should not be deemed to limit the scope of the disclosure in any way. Additionally, features described in connection with the various or specific embodiments are not to be construed as not appropriate for use in connection with other embodiments disclosed herein unless such exclusivity is explicitly stated or implicit from context.

### Definitions

The term “about” generally refers to plus or minus 10% of the indicated number. For example, “about 10%” may indicate a range of 9% to 11%, and “about 20” may mean from 18-22. Other meanings of “about” may be apparent from the context, such as rounding off; for example, “about 1” may also mean from 0.5 to 1.4.

The term “encodes” and the phrase “codes for” refer to the ability of a nucleotide sequence or an amino acid sequence to provide information that describes the sequence of nucleotides or amino acids in another sequence or in a molecule. Thus, a nucleotide sequence encodes a molecule that contains the same nucleotides as in the nucleotide sequence that encodes it; that contains the complementary nucleotides according to Watson-Crick base pairing rules; that contains the RNA equivalent of the nucleotides that encode it; that contains the RNA equivalent of the complement of the nucleotides that encode it; that contains the amino acid sequence that can be generated based on the consecutive codons in the sequence; and that contains the amino acid sequence that can be generated based on the complement of the consecutive codons in the sequence. The phrase “coded by” means that the sequence of a first molecule such as a polypeptide is determined by the code of a second molecule such as a polynucleotide.

Throughout this specification, the word “comprise” or variations such as “comprises” or “comprising” will be understood to imply the inclusion of a stated integer (or component) or group of integers (or components), but not the exclusion of any other integer (or components) or group of integers (or components).

The term “including” is used to mean “including but not limited to.” “Including” and “including but not limited to” are used interchangeably.

Any example(s) following the term “e.g.” or “for example” is not meant to be exhaustive or limiting.

The term “cargo”, as used throughout this specification, refers to the genetic material present in the polynucleotide of the disclosure and that is cut and inserted into the host DNA. The cargo region may be integrated into a host's DNA through random integration or may be integrated via more targeted systems, such as systems that make use of transposon vectors or viral technologies.

An “expression cassette”, as used herein, refers to a polynucleotide comprising a gene and regulatory sequences to be expressed by a transfected cell. The expression cassette comprises a gene that encodes protein(s) to be delivered to a cell or tissue, as well as regulatory elements controlling expression of encoded protein(s). Regulatory elements include, but are not limited to, promoters, enhancers, terminator sequences, 3′ untranslated regions, such as polyadenylation sequences, and the like, mRNA stability sequences (e.g. Woodchuck Hepatitis Virus Posttranscriptional Regulatory Element; WPRE), sequences that allow for internal ribosome entry sites (IRES) of bicistronic mRNA, sequences necessary for episome maintenance (e.g., sMARs), sequences that avoid or inhibit viral recognition by Toll-like or RIG-like receptors and/or sequences necessary for transduction into cells.

A “cloning cassette,” as used herein, refers to a polynucleotide comprising (i) a multiple cloning site for the introduction of an open reading frame or gene; (ii) a promoter sequence to control the expression of the gene and (iii) a 3′ UTR, such as a polyadenylation sequence.

A “selection cassette,” as used herein, refers to a polynucleotide encoding a selection marker that is used to identify if the gene of interest has been successfully transfected and integrated into the cell.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.), multi-specific antibodies (e.g., bi-specific, tri-specific antibodies) that specifically bind to a given antigen/s. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds.

The term “antibody,” as used herein, also includes the term “antigen binding fragment,” which refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments.

Headers are provided for the convenience of the reader and do not limit the scope of any of the embodiments disclosed herein.

### Polynucleotides and Vectors

The polynucleotides of the present disclosure may be single stranded or double stranded or combinations thereof. Further, they may comprise, consist essentially, or consist of ribonucleic acids, deoxyribonucleic acids, and combinations thereof. One or more, if not all of the nucleotides, may be modified (e.g., 2-′O-methyl or LNA modified). Alternatively, one or more, if not all of the nucleotides, may be unmodified.

In some embodiments, the polynucleotide of the disclosure comprises a selection cassette. In some embodiments, the polynucleotide of the disclosure comprises a selection cassette and one or more cloning cassettes. In some embodiments, the polynucleotide of the disclosure comprises a selection cassette and 2, 3, 4, 5, 6, 7, 8 or more cloning cassettes. In some embodiments, the polynucleotide comprises one or more expression cassettes and at least one selection cassette. In some embodiments, the polynucleotide of the disclosure comprises a selection cassette and 2, 3, 4, 5, 6, 7, 8 or more expression cassettes. By way of non-limiting examples, there may be one expression cassette and one selection cassette; or two expression cassettes and one selection cassette; or three expression cassettes and one selection cassette; or four expression cassettes and one selection cassette. In some embodiments, all of the cassettes are oriented is the same direction, whereas in other embodiments, all of the expression cassettes are (or the single expression cassette when there is only one) is oriented in one direction (a forward direction), while the selection cassette is oriented in the opposite (reverse) direction. In still other embodiments in which there are at least two expression cassettes, one or more expression cassettes are oriented in a forward direction and the other expression cassette or plurality of cassettes, as well as the selection cassette are oriented in the reverse direction. In still other embodiments, one expression cassette or a plurality of expression cassettes, as well as the selection cassette, are oriented in a forward direction and the other expression cassette, or a plurality of expression cassettes are oriented in the reverse direction. When cassettes are in different orientations, the RNA polymerases will use different strands of the two strands of the double stranded polynucleotide as templates. By way of a non-limiting example, each cassette can be between 0.3 kb and 10 kb long. Because the polynucleotides may be either single stranded or double stranded, the lengths may be defined by the number of nucleotides or base pairs respectively.

The polynucleotides may, in some embodiments, be or be part of vectors such as transposon vectors, lentiviral vectors, or retroviral vectors. In some embodiments, the polynucleotides of the disclosure are part of a vector selected from the group consisting of transposon vectors, lentiviral vectors, retroviral vectors, adeno-associated viral vectors, adenoviral vectors and herpes simplex viral vectors. Additionally, or alternatively, they may be or be part of linear or circular molecules such as plasmids. Within the polynucleotides is a cargo region that may be integrated into a host's DNA through random integration or through more targeted systems for integration such as systems that make use of transposon or viral technologies.

In some embodiments, the present disclosure is directed to vectors. In some embodiments, the polynucleotide of the disclosure is comprised within a vector. Examples of vectors that may be used in the present disclosure include, but are not limited to, transposon vectors, lentiviral vectors, retroviral vectors, adeno-associated viral vectors, adenoviral vectors, herpes simplex viral vectors, etc.

The vectors may, for example, be transposon vectors or random integration vectors, which differ from transposon vectors in that random integration vectors lack ITRs repeat sequences. The vectors may comprise, consist essentially of, or consist of polynucleotides or the present disclosure.

In some embodiments, the present disclosure is directed to transposon vectors. These vectors are double stranded DNA sequences that in some embodiments comprise an expression cassette and a selection cassette, or comprise a first expression cassette, a second expression cassette and a selection cassette. Alternatively, the vectors of the disclosure comprise one or more expression cassettes and one or more selection cassettes. Collectively, cassettes within a vector form the cargo, which may also be referred to as the cargo region or gene(s) of interest (GOI) region. Within a selection cassette, there may, for example, be a stretch of polynucleotides that code for a protein or other detectable moiety and that is under the control of a promoter region. This moiety may be termed a selection marker and the region that codes for it may be termed a selection marker sequence.

The transposon vector may be configured to be part of a Tc1/mariner system. Thus, a Tc1/mariner transposase protein (alone or in combination with other known factors for the Tc1/mariner system) is capable of catalyzing translocation of the cargo from the transposon into a nucleotide sequence in a host cell, also referred to as host's nucleotide or DNA sequence (which also may be referred to as integrating it). Thus, the cargo region may comprise or be flanked by one or a pair of sequences that are recognized by a Tc1/mariner transposase.

The transposon vector may be linear or circular, e.g., in the form of plasmid. Within the transposon vector, the cargo is located between a pair of inverted terminal repeat (“ITR”) sequences. In some embodiments, within each ITR region is a direct repeat (“DR”) sequence or a pair of DR sequences. By way of non-limiting examples, in some embodiments, each ITR is about 200-250 base pairs in length, and if present, within each ITR there may be a DR that is about 15 to 35 base pairs in length. In some embodiments, when the cargo region is integrated into the host DNA, which may be either genomic DNA or extrachromosomal DNA, a DR sequence is juxtaposed to the host DNA at each end of the cargo region. Examples of inverted repeat sequences and direct repeat sequences that are known for use in connection with Sleeping Beauty transposons, which are types of Tc1/mariner transposons, are provided in WO 03/089618, Transposon System and Methods of Use, published Oct. 30, 2003, which is incorporated by reference in its entirety.

In some embodiments, the vector comprises at least one expression cassette and at least one selection cassette. By way of a non-limiting example, within the transposon vector, a first expression cassette, and a second expression cassette may be oriented in the same direction, which may be referred to as a first direction, while the selection cassette is oriented in a second direction that is the opposite of the first direction. For example, the first expression cassette and the second expression cassette may be oriented in forward direction while the selection cassette may be oriented in a reverse direction. Alternatively, all cassettes may be oriented in the same direction, or the first expression cassette may be oriented in a forward direction and both the second expression cassette, and the selection cassette may be oriented in the reverse direction, or the second expression cassette may be oriented in a forward direction and both the first expression cassette, and the selection cassette may be oriented in the reverse direction.

The selection cassette of the polynucleotide or vector of the disclosure may comprise a promoter sequence that is a truncated version of a Herpes Simplex Virus (HSV) thymidine kinase (TK) promoter sequence or a derivative of a truncated version of an HSV promoter sequence. The truncated version of HSV-TK promoter may be referred to herein as HSVMin and their sequences may be referred to as HSVMin promoter sequences.

The HSVMin promoter wild-type (WT) sequence fragment is provided as SEQ ID NO: 1 in Table 3 below. In some embodiments, the HSVMin promoter sequence is no more than 240 nucleotides long, no more than 220 nucleotides long, no more than 200 nucleotides long, no more than 180 nucleotides long, no more than 160 nucleotides long, or no more than 140 nucleotides long. In some embodiments, the HSVMin sequence is 100 to 180 nucleotides long or 120 to 160 nucleotides long or about 140 nucleotides long.

In some embodiments, the HSVMin promoter sequence comprises a nucleotide sequence that comprises at least 80% or at least 85% or at least 90% or at least 95% or 100% sequence identity to a sequence selected from the group consisting of any of the sequences SEQ ID NO: 2 to SEQ ID NO: 25, over a span of at least 100 nucleotides, at least 120 nucleotides, or at least 140 nucleotides. In some embodiments, the HSVMin promoter sequence comprises a nucleotide sequence that comprises at least 80% or at least 85% or at least 90% or at least 95% or 100% sequence identity to a sequence complementary to any of the sequences SEQ ID NO: 2 to SEQ ID NO: 25, over a span of at least 100 nucleotides, at least 120 nucleotides, or at least 140 nucleotides. In some embodiments, the HSVMin promoter sequence is selected from the group consisting of any one of the sequences SEQ ID NO: 2 to SEQ ID NO: 25. In one embodiment, the HSVMin sequence comprises at least 80% or at least 85% or at least 90% or at least 95% or 100% sequence identity to SEQ ID NO: 14 or to a sequence that is complementary to SEQ ID NO: 14. In one embodiment, the HSVMin sequence comprises at least 80% or at least 85% or at least 90% or at least 95% or 100% sequence identity to SEQ ID NO: 24 or to a sequence that is complementary to SEQ ID NO: 24. In one embodiment, the HSVMin sequence comprises at least 80% or at least 85% or at least 90% or at least 95% or 100% sequence identity to SEQ ID NO: 14. In one embodiment, the HSVMin sequence comprises at least 80% or at least 85% or at least 90% or at least 95% or 100% sequence identity to SEQ ID NO: 24. In some embodiments, the use of any of the polynucleotides of the disclosure including the sequence of a HSVMin promoter comprising at least 80% or at least 85% or at least 90% or at least 95% or 100% sequence identity to SEQ ID NO: 14 or SEQ ID NO: 24 or a sequence that is complementary to SEQ ID NO: 14 or SEQ ID NO: 24 leads to a stringent selection process. This stringent selection can lead to the selection of cells that have integrated a higher number of copies of the selection cassette and, therefore, a higher number of copies of the expression cassette.

Sequence identity between two similar sequences can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol.48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al., 1990, “Basic Local Alignment Search Tool,” J. Mol. Biol. 215:403-10, the “BLAST” algorithm, see blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

Optionally, the sequence identity is determined over a region that is at least about 50 nucleotides in length, or in some cases over a region that is 100 nucleotides in length.

The selection cassette of the polynucleotide or vector of the disclosure also contains a selection marker sequence, e.g., a sequence of a metabolic marker as such that can be used to identify if the gene of interest has been successfully transfected. Examples of suitable selection markers include, but are not limited to, a coding sequence for a drug resistance protein or a metabolic gene. Examples of suitable selection markers include, but are not limited, a glutamine synthetase (GS) sequence or derivatives thereof, a dihydrofolate reductase sequence or derivatives thereof, a neomycin phosphotransferase (neol) sequence, hygromycin B phosphotransferase (hyg1) sequence, puromycin-N-acetyltransferase (purol) sequence, blasticidin S deaminase (bsri) sequence, xanthine/guanine phosphoribosyl transferase (gptsequence), and herpes simplex virus thymidine kinase (HSV-k) sequence, or derivatives thereof.

Methods to identify the number of gene copies integrated into the host cell DNA are known in the art. Examples of such methods include, but are not limited to, droplet digital PCR (ddPCR) and qPCR. For example, in some embodiments, the number of selection marker integrated into the host cell DNA is determined using ddPCR. The analysis of the selection marker copy number indicates the number of copies integrated of the gene of interest.

In some embodiments, the selection marker sequence of the polynucleotide or vector of the disclosure comprises a GS sequence. The glutamine synthetase sequence codes for a glutamine synthetase protein. In some embodiments, the glutamine synthetase sequence is from Cricetulus griseus and comprises the sequence below set forth in SEQ ID NO: 26:

ATGGCCACCTCAGCAAGTTCCCACTTGAACAAAAACATCAAGCAAATG
TACTTGTGCCTGCCCCAGGGTGAGAAAGTCCAAGCCATGTATATCTGG
GTTGATGGTACTGGAGAAGGACTGCGCTGCAAAACCCGCACCCTGGAC
TGTGAGCCCAAGTGTGTAGAAGAGTTACCTGAGTGGAATTTTGATGGC
TCTAGTACCTTTCAGTCTGAGGGCTCCAACAGTGACATGTATCTCAGC
CCTGTTGCCATGTTTCGGGACCCCTTCCGCAGAGATCCCAACAAGCTG
GTGTTCTGTGAAGTTTTCAAGTACAACCGGAAGCCTGCAGAGACAAAT
TTAAGGCACTCGTGTAAACGGATAATGGACATGGTGAGCAACCAGCAC
CCCTGGTTTGGAATGGAACAGGAGTATACTCTGATGGGAACAGATGGG
CACCCTTTTGGTTGGCCTTCCAATGGCTTTCCTGGGCCCCAAGGTCCG
TATTACTGTGGTGTGGGCGCAGACAAAGCCTATGGCAGGGATATCGTG
GAGGCTCACTACCGCGCCTGCTTGTATGCTGGGGTCAAGATTACAGGA
ACAAATGCTGAGGTCATGCCTGCCCAGTGGGAGTTCCAAATAGGACCC
TGTGAAGGAATCCGCATGGGAGATCATCTCTGGGTGGCCCGTTTCATC
TTGCATCGAGTATGTGAGGACTTTGGGGTAATAGCAACCTTTGACCCC
AAGCCCATTCCTGGGAACTGGAATGGTGCAGGCTGCCATACCAACTTT
AGCACCAAGGCCATGCGGGAGGAGAATGGTCTGAAGCACATCGAGGAG
GCCATCGAGAAACTAAGCAAGCGGCACAGGTACCACATTCGAGCCTAC
GATCCCAAGGGGGGCCTGGACAATGCCCGTCGTCTGACTGGGTTCCAC
GAAACGTCCAACATCAACGACTTTTCTGCTGGTGTCGCCAATCGCAGT
GCCAGCATCCGCATTCCCCGGACTGTCGGCCAGGAGAAGAAAGGTTAC
TTTGAAGATCGCCGCCCCTCTGCCAATTGTGACCCCTTTGCAGTGACA
GAAGCCATCGTCCGCACATGCCTTCTCAATGAGACTGGCGACGAGCCC
TTCCAATACAAAAACTAATGA

In some embodiments, the glutamine synthetase sequence is the Homo sapiens glutamine synthetase sequence (PubMed ID accession number: 29642388), SEQ ID NO: 31:

ATGACCACCTCAGCAAGTTCCCACTTAAATAAAGGCATCAAGCAGGTG
TACATGTCCCTGCCTCAGGGTGAGAAAGTCCAGGCCATGTATATCTGG
ATCGATGGTACTGGAGAAGGACTGCGCTGCAAGACCCGGACCCTGGAC
AGTGAGCCCAAGTGTGTGGAAGAGTTGCCTGAGTGGAATTTCGATGGC
TCCAGTACTTTACAGTCTGAGGGTTCCAACAGTGACATGTATCTCGTG
CCTGCTGCCATGTTTCGGGACCCCTTCCGTAAGGACCCTAACAAGCTG
GTGTTATGTGAAGTTTTCAAGTACAATCGAAGGCCTGCAGAGACCAAT
TTGAGGCACACCTGTAAACGGATAATGGACATGGTGAGCAACCAGCAC
CCCTGGTTTGGCATGGAGCAGGAGTATACCCTCATGGGGACAGATGGG
CACCCCTTTGGTTGGCCTTCCAACGGCTTCCCAGGGCCCCAGGGTCCA
TATTACTGTGGTGTGGGAGCAGACAGAGCCTATGGCAGGGACATCGTG
GAGGCCCATTACCGGGCCTGCTTGTATGCTGGAGTCAAGATTGCGGGG
ACTAATGCCGAGGTCATGCCTGCCCAGTGGGAATTTCAGATTGGACCT
TGTGAAGGAATCAGCATGGGAGATCATCTCTGGGTGGCCCGTTTCATC
TTGCATCGTGTGTGTGAAGACTTTGGAGTGATAGCAACCTTTGATCCT
AAGCCCATTCCTGGGAACTGGAATGGTGCAGGCTGCCATACCAACTTC
AGCACCAAGGCCATGCGGGAGGAGAATGGTCTGAAGTACATCGAGGAG
GCCATTGAGAAACTAAGCAAGCGGCACCAGTACCACATCCGTGCCTAT
GATCCCAAGGGAGGCCTGGACAATGCCCGACGTCTAACTGGATTCCAT
GAAACCTCCAACATCAACGACTTTTCTGCTGGTGTAGCCAATCGTAGC
GCCAGCATACGCATTCCCCGGACTGTTGGCCAGGAGAAGAAGGGTTAC
TTTGAAGATCGTCGCCCCTCTGCCAACTGCGACCCCTTTTCGGTGACA
GAAGCCCTCATCCGCACGTGTCTTCTCAATGAAACCGGCGATGAGCCC
TTCCAGTACAAAAAT.

In some embodiments, the glutamine synthetase sequence is the M. musculus glutamine synthetase sequence (PubMed ID accession number: 2475638), SEQ ID NO: 32:

ATGGCCACCTCAGCAAGTTCCCACTTGAACAAAGGCATCAAGCAAATG
TACATGTCCCTGCCCCAGGGTGAGAAAGTCCAAGCCATGTATATCTGG
GTTGATGGTACCGGAGAAGGACTGCGCTGCAAGACCCGTACCCTGGAC
TGTGAGCCCAAGTGTGTGGAAGAGTTACCTGAGTGGAACTTTGATGGC
TCTAGTACCTTTCAGTCTGAAGGCTCCAACAGCGACATGTACCTCCAT
CCTGTTGCCATGTTTCGAGACCCCTTCCGCAGAGACCCCAACAAGCTG
GTGCTATGTGAAGTTTTCAAGTATAACCGGAAACCTGCAGAGACCAAC
TTGAGGCACATCTGTAAACGGATAATGGACATGGTGAGCAACCAGCAC
CCCTGGTTTGGAATGGAGCAGGAATATACTCTTATGGGAACAGACGGC
CACCCATTTGGTTGGCCTTCCAATGGCTTCCCTGGACCCCAAGGCCCG
TATTACTGCGGTGTGGGAGCAGACAAGGCCTACGGCAGGGACATCGTG
GAGGCTCACTACCGGGCCTGCTTGTATGCTGGAGTCAAGATTACGGGG
ACAAATGCGGAGGTTATGCCTGCCCAGTGGGAATTCCAAATAGGACCC
TGTGAGGGGATCCGAATGGGAGATCATCTTTGGATAGCCCGTTTTATC
TTGCATCGGGTGTGCGAAGACTTTGGGGTGATAGCAACCTTTGACCCC
AAGCCCATTCCAGGGAACTGGAATGTTGCAGGCTGCCATACCAACTTC
AGCACCAAGGCCATGCGGGAGGAGAATGGTCTGAAGTGCATTGAGGAG
GCCATTGACAAACTGAGCAAGAGGCACCAGTACCACATTCGCGCCTAC
GATCCCAAGGGGGGCCTGGACAATGCCCGTGCTCTGACTGGATTCCAC
GAAACCTCCAACATCAACGACTTTTCTGCTGGTGTTGCCAACCGCGGT
GCCAGTATCCGCATTCCCCGGACTGTCGGCCAGGAGAAGAAGGGCTAC
TTTGAAGACCGTCGCCTTCGTGCCAATTGTGACCCCTATGCGGTGACA
GAAGCCATCGTCCGCACGTGTCTCCTCAACGAAACAGGCGACGAACCC
TTCCAATACAAGAACTAAGTGA;

In some embodiments, the glutamine synthetase sequence is the Onychomys torridus glutamine synthetase sequence (PubMed ID accession number: XM_036202125), SEQ ID NO: 33:

ATGGCCACCTCAGCAAGTTCCCACTTGAACAAAGGCATCAAGCAAATG
TACATGTCCCTGCCCCAGGGTGAGAAAGTGCAAGCCATGTATATCTGG
GTGGACGGTACCGGAGAAGGACTGCGTTGTAAGACCCGCACCCTGGAC
TGTGAGCCCAAGTGTGTCGAAGAGTTACCTGAGTGGAATTTTGATGGA
TCTAGTACCTTTCAGTCCGAGGGCTCCAACAGTGACATGTATCTCAGC
CCTGTTGCCATGTTTCGGGACCCCTTCCGCAAAGAGCCCAACAAGTTG
GTGTTCTGTGAAGTTTTCAAGTACAACCGGAAGCCTGCAGAGACCAAT
TTAAGACACACCTGTAAACGGATAATGGACATGGTGAGCAGCCAGCAC
CCCTGGTTTGGAATGGAACAGGAATACACTCTCATGGGAACAGATGGG
CACCCTTTTGGTTGGCCATCCAATGGCTTCCCTGGGCCCCAAGGTCCA
TATTACTGTGGCGTGGGAGCAGACAAAGCCTATGGCAGGGATATTGTG
GAGGCCCACTACCGGGCCTGCTTGTATGCTGGAGTCAAGATTACAGGA
ACAAATGCTGAGGTCATGCCTGCCCAGTGGGAATTCCAGATAGGACCG
TGTGAAGGAATCCGCATGGGAGATCATCTCTGGGTGGCCCGGTTCATC
TTGCATCGCGTATGTGAAGACTTTGGGGTGATAGCAACCTTTGACCCC
AAGCCCATTCCTGGGAACTGGAATGGCGCAGGCTGCCATACCAACTTT
AGCACCAAGGCCATGCGGGAGGAGAACGGTCTGAAGTACATTGAGGAG
GCCATTGAGAAACTGAGCAAGCGGCACCAGTACCACATTCGCGCCTAC
GATCCCAAGGGGGGCCTGGACAACGCCCGGCGTCTGACTGGATTCCAC
GAAACCTCCAACATCAACGACTTTTCTGCCGGCGTGGCCAACCGCAGC
GCCAGCATCCGCATTCCCCGGACTGTCGGCCAGGAGAAGAGGGGTTAC
TTCGAAGACCGTCGTCCTTCTGCCAACTGTGACCCGTTTGCCGTGACA
GAAGCCATCGTCCGCACATGCCTTCTCAACGAGACTGGCGACGAGCCC
TTCCAGTACAAGAACTAA

Within the scope of the present disclosure are selection cassettes that comprise selection marker sequences that comprise at least 80%, at least 85%, at least 90%, at least 95% or 100% sequence identity to SEQ ID NO: 26 over 900 consecutive nucleotides. Within the scope of the present disclosure are selection cassettes that comprise selection marker sequences that comprise at least 80%, at least 85%, at least 90%, at least 95% or 100% sequence identity to a sequence complementary to SEQ ID NO: 26 over 900 consecutive nucleotides.

Within the scope of the present disclosure are selection cassettes that comprise selection marker sequences that comprise at least 80%, at least 85%, at least 90%, at least 95% or 100% sequence identity to SEQ ID NO: 31 over 900 consecutive nucleotides. Within the scope of the present disclosure are selection cassettes that comprise selection marker sequences that comprise at least 80%, at least 85%, at least 90%, at least 95% or 100% sequence identity to a sequence complementary to SEQ ID NO: 31 over 900 consecutive nucleotides.

Within the scope of the present disclosure are selection cassettes that comprise selection marker sequences that comprise at least 80%, at least 85%, at least 90%, at least 95% or 100% sequence identity to SEQ ID NO: 32 over 900 consecutive nucleotides. Within the scope of the present disclosure are selection cassettes that comprise selection marker sequences that comprise at least 80%, at least 85%, at least 90%, at least 95% or 100% sequence identity to a sequence complementary to SEQ ID NO: 32 over 900 consecutive nucleotides.

Within the scope of the present disclosure are selection cassettes that comprise selection marker sequences that comprise at least 80%, at least 85%, at least 90%, at least 95% or 100% sequence identity to SEQ ID NO: 33 over 900 consecutive nucleotides. Within the scope of the present disclosure are selection cassettes that comprise selection marker sequences that comprise at least 80%, at least 85%, at least 90%, at least 95% or 100% sequence identity to a sequence complementary to SEQ ID NO: 33 over 900 consecutive nucleotides.

In some embodiments, the selection cassette may also comprise a polyadenylation sequence. Examples of polyadenylation sequences include, but are not limited to, the SV40 polyadenylation sequence, the signal rabbit β-globin polyadenylation sequence, the bovine growth hormone polyadenylation sequence, or another suitable heterologous or endogenous polyadenylation sequences known in the art. In some embodiments, the polyadenylation sequence comprises the SV40 polyadenylation sequence, SEQ ID NO: 27:

AACTTGTTTATTGCAGCTTATAATGGTTACAAATAAAGCAATAGCATC
ACAAATTTCACAAATAAAGCATTTTTTTCACTGCATTCTAGTTGTGGT
TTGTCCAAACTCATCAATGTATCTTA.

In some embodiments, the polyadenylation sequence comprises the bovine growth hormone polyadenylation sequence, SEQ ID NO: 28:

CTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAA
ATGAGGAAATTGCATCGCATTGTCTGAGTAGGTGTCATTCTATTCTGG
GGGGTGGGGTGGGGCAGGACAGCAAGGGGGAGGATTGGGAAGACAATA
GCAGGCATGCTGGGGATGCGGTGGGCTCTATGG.

Within the selection cassette, the selection marker sequence may be located between the HSVMin promoter sequence and the polyadenylation sequence.

In some embodiments, within the selection cassette, the glutamine synthetase sequence is located between the HSVMin promoter sequence and the polyadenylation sequence.

Each cloning cassette or expression cassette may also comprise a multiple cloning site that is located downstream of the promoter. The multiple cloning site is a stretch of DNA that may contain one more restriction sites. In some embodiments, the multiple cloning site is MSC1: 5′-GTCGACGCCGCCACCATGAGAGACGTCTAAGGTAACGTCTCGTAATGAG CGGCCGC-3′ (SEQ ID NO: 29). In other embodiments, the multiple cloning site is MSC2: 5′-TCTAGACTAAGCCGCCACCAGAGACGTACTAAGTTACGTCTCTCGATGAG AATTC-3′ (SEQ ID NO: 30). In some embodiments, the multiple cloning site is MCS3: 5′-GTCGACGCATACGGAAATGCATATTCATCGTATCGCGATATTGTGTCTAT CGATATTCACCGAGCGGCCGC-3′ (SEQ ID NO: 39). In other embodiments, the multiple cloning site is MCS4: 5′-TCTAGACTATTCCGCTGATCATTGCTTACACACGTGCGGTACGTCGGGCG CGCCTGGGCATACGGAACCCCGGGACGTCTCTCGATA-3′ (SEQ ID NO: 40). In other embodiments, the multiple cloning site is MSC5: 5′ GTCGACGCATACGGAACCTAGGATTCATCGTATCGCGATATTGTGTCTAT CGATATTCACCGAGCGGCCGC-3′ (SEQ ID NO: 43).

The promoter sequences of different cassettes may be the same or different. Examples of promoter sequences that may be used within the cloning cassette or expression cassettes or post-translational modification cassette include, but are not limited to, a SV40 promoter or a fragment thereof, an EF1α promoter or a fragment thereof, a CMV promoter or a fragment thereof, a PGK promoter or a fragment thereof, a CAG promoter or a fragment thereof, and a β-globin promoter or a fragment thereof.

Optionally, each cloning cassette or expression cassette may comprise a polyadenylation sequence downstream of the multiple cloning site. Examples of polyadenylation sequences include, but are not limited to, the SV40 polyadenylation sequence and the bovine growth hormone polyadenylation sequence.

In some embodiments, each expression cassette contains a protein coding sequence. Each protein coding sequence may, for example, be located between the promoter and polyadenylation sequence within the multiple cloning site sequence. By way of a non-limiting example, a first expression cassette may comprise a nucleotide sequence that codes for a polypeptide such as an antibody heavy chain and a second expression cassette may comprise a nucleotide sequence that codes for a polypeptide such as an antibody light chain. By way of another non-limiting example, the first expression cassette may comprise a nucleotide sequence that codes for an antibody light chain and the second expression cassette comprises a nucleotide sequence that codes for an antibody heavy chain. By way of another non-limiting example, the polynucleotide of the disclosure comprises only one expression cassette that codes for a protein and a selection cassette.

Examples of heavy chain antibody coding sequences and light chain antibody coding sequences, include but are not limited to nucleotide sequences that code for IgGs, e.g., an IgG1, an IgG2, and IgG3 or IgG4. Examples of antibody coding sequences that may be included in the expression cassette include, but are not limited to, Trastuzumab, Nivolumab, Pembrolizumab, Denosumab, Ocrelizumab, Secukinumab, Tocilizumab, Blinatumumab, Vanucizumab and Rituximab. Examples of coding sequences of other molecules for the expression cassettes include, but are not limited to, nucleotide sequences that encode for Etanercept, Epoetin Alfa, cytokines (such as Interferon), hormones (such as Gonadotropins) or Octocog alpha.

In other embodiments in which there are two or more expression cassettes, only one of the first expression cassette and the second expression cassette comprises a protein coding sequence (while also comprising a promoter sequence, a multiple cloning site, and a polyadenylation sequence), while the other only contains a scaffold, e.g., one, two or all three of: a promoter sequence, a multiple cloning site, and a polyadenylation sequence, but no protein coding sequences. When there is only one expression cassette, it may or may not have a protein coding region.

In some embodiments, one or more additional expression cassettes are present, e.g., a third expression cassette, a fourth expression cassette, a fifth expression cassette, etc. These additional expression cassettes may be part of the same polynucleotide or vector as the selection cassette or part of a separate polynucleotide or vector. When the selection cassette is between the first expression cassette and the second expression cassette, any additional expression cassettes that are part of the same polynucleotide or vector may be between the first expression cassette and the selection cassette or between the second expression cassette and the selection cassette, or distal to the selection cassette such that either the first expression cassette and the second expression cassette are between the third expression cassette and the selection cassette.

In some embodiments, there is a third expression cassette that is part of the same polynucleotide or vector as the first expression cassette and the second expression cassette.

In some embodiments, the expression cassette is a “post-translational modification cassette”. The “post-translational modification cassette” comprises a sequence that codes for a polypeptide that is capable of causing a post-translational modification to another polypeptide. The “another polypeptide” can be part of the same polynucleotide or vector or be in a separate polynucleotide or vector. Therefore, in some embodiments, the polynucleotide of the disclosure comprises (i) one or more expression cassettes encoding for one or more polypeptides; a post-translational modification cassette encoding for a polypeptide that is capable of causing a post-translational modification to the one or more polypeptides of (i); and (iii) a selection cassette.

In some embodiments, one of the one or more expression cassettes is a “post-translational modification cassette,” which comprises a sequence that codes for a polypeptide that is capable of causing a post-translational modification to another polypeptide.

The post-translational modification cassette may, for example, code for a polypeptide that causes a post-translational modification of one or more of the polypeptides, including but not limited to full proteins and fragments thereof, coded by nucleotide sequences that are contained within the first expression cassette and/or the second expression cassette or the more expression cassettes comprised within the polynucleotide or vector of the disclosure. The use of a post-translational modification cassette that codes for polypeptides, such as enzymes with this functionality, may be particularly advantageous in biotherapeutic applications. An example of this type of post-translational modification is glycosylation. In some embodiments, the post-translational modification comprises a polynucleotide that codes for a glycosyltransferase. Examples of glycosyltransferases include, but are not limited to, sialyltransferase, galactosyltransferase, fucosyltransferase, etc. Examples of glycosyltransferases are provided in Table 1 of Nguyen, N. T. B., Lin, J., Tay, S. J. et al. Sci Rep 11, 12969 (2021), which is herein incorporated by reference.

In some embodiments, the glycosyltransferase is a sialyltransferase. Examples of sialyltransferases include, but are not limited to, ST3Gal4, ST3Gal5, ST3Gal6, ST6Gal1 and ST6Gal2. Thus, in one embodiment, the post-translational modification cassette codes for ST6Gal1 (ST6 beta-galactoside alpha-2,6-sialyltransferase 1) or ST6Gal2 (ST6 beta-galactoside alpha-2,6-sialyltransferase 2), or the catalytic domain of ST6Gal1 or ST6Gal2, or a truncated and/or modified versions of ST6Gal1 or ST6Gal2. A nucleotide sequence that codes for ST6Gal1 is publicly available at: https://www.ncbi.nlm.nih.gov/nuccore/NM_001353916 (Accession number NM_001353916) or https://www.ncbi.nlm.nih.gov/nuccore/NM_003032 (Accession number NM_003032.3), the disclosure of which is incorporated by references as is set forth fully herein. A nucleotide sequence that codes for ST6Gal2 is publicly available at: https://www.ncbi.nlm.nih.gov/nuccore/NM_001142351 (Accession number NM_001142351), the disclosure of which is incorporated by reference as is set forth fully herein.

In some embodiments, the selection cassette is not between expression cassettes. Thus, two or more expression cassettes may be adjacent to each other followed by the selection cassette, which may be in the same or a different orientation from the expression cassettes when all of the expression cassettes are in the same orientation.

The polynucleotides or vectors of the disclosure may also be designed to include sites that are recognized by restriction enzymes. For example, restriction enzyme sites may be found before, between or after an expression or selection cassette. Furthermore, restriction enzyme sites may be located between different elements, which allows for the selective modification of a transposon sequence. For example, a restriction site between two cassettes can be used to introduce regulatory elements.

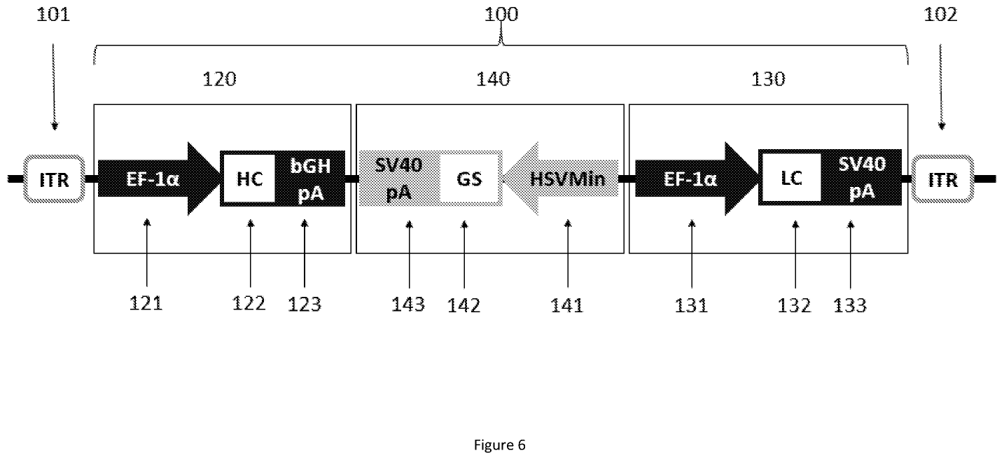

FIG. 6 provides a linear representation of a transposon vector with a gene of interest (“GOI”) region 100, between a first ITR 101 and a second ITR 102, in which the first ITR is upstream of both the GOI and the second ITR. As shown, within the GOI region, there are a first expression cassette 120, a second expression cassette 130, and a selection cassette 140, with the selection cassette being located between the first expression cassette and the second expression cassette. Also as shown, the first expression cassette is located between the first ITR and the selection cassette, while the second expression cassette is located between the selection cassette and the second ITR. Further, the first expression cassette and the second expression cassette are oriented in a first direction, e.g., a forward direction, while the selection cassette is oriented in a second direction that is opposite to the first direction, e.g., a reverse direction. Spacer sequences can be located between the cassettes or between the elements, and the spacer sequences may range from 0 to 150 bp (or 0-150 nucleotides in single-stranded molecules). Such spacer sequences are non-regulatory sequences and can be used as a buffer sequence and/or to introduce additional restriction sites. For example, a restriction site between an expression cassette can be used to introduce regulatory elements, such as MAR elements, UCOES, and insulators.

Within the first expression cassette is a first promoter, e.g., EF1α 121 or CMV (not shown). Downstream of the promoter is a coding region, e.g., a heavy chain antibody coding region (“HC”) 122, followed by a polyadenylation sequence, e.g., a bovine growth hormone (“bGH”) polyadenylation sequence (“bGH pA”) 123.

Within the second expression cassette is a second promoter, e.g., EFla or CMV 131. Downstream of the promoter is a coding region, e.g., a light chain antibody coding region (“LC”) 132, followed by a polyadenylation sequence, e.g., SV40 polyadenylation sequence (“SV40 pA”) 133.

Within the selection cassette, which is oriented in the opposite direction of the first and second expression cassettes, is a third promoter, HSVMin 141. Downstream of the promoter is a coding region, e.g., glutamine synthetase (“GS”) 142, followed by a polyadenylation sequence, e.g., SV40 polyadenylation sequence (“SV40 pA”) 143. Notably, between the first promoter (which is in the first expression cassette) and the third promoter are the coding region (HC) and polyadenylation sequence of the first expression cassette and the coding region (GS) and polyadenylation sequence of the selection cassette, while between the second promoter (which is in the second expression cassette) and the third promoter there is an absence of coding regions (LC) and polyadenylation sequence of the second expression cassette and the coding region (GS) and polyadenylation sequence.

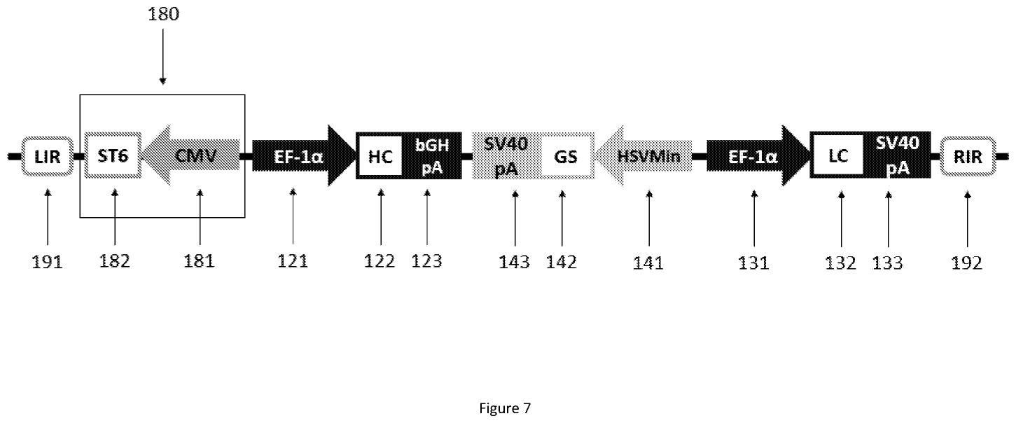

FIG. 7 depicts a variation of the design that is depicted in FIG. 6. In FIG. 7, a third expression cassette or post-translational modification cassette is present between the first ITR, labeled as LIR to denote that it is the left independent repeat 191 and the EF-1α promoter 121 of the first expression cassette. The third expression cassette, including its promoter (e.g., CMV as shown), may be oriented in the same direction as the first expression cassette, or as shown in FIG. 7 in the opposite direction. Thus, as shown, the directionality of the cassettes alternates: the first expression cassette, which as shown contains elements 121 (EF-1α promoter), 122 (HC), and 123 (bGH pA) and the second expression cassette, which as shown contains elements 131 (EF-1α promoter), 132 (LC), and 133 (SV40 pA) are oriented in one direction and the third expression cassette or post-translational modification cassette and the selection cassette are oriented in the opposite direction. In this Figure, the third expression cassette is downstream of the selection cassette when looked at from their directionality. In alternative embodiments, the third expression cassette may be oriented in the same direction as one or both of the first expression cassette and the second expression cassette, one or both of which may be in the same or opposite orientation to that of the selection cassette.

In some embodiments, the third expression cassette or post-translational modification cassette as shown codes for and is capable of expressing ST6Gal1 or ST6Gal2 (labeled generally as ST6 in the Figure) or a fragment or modified version thereof 182 and is under its own promoter, which by way of example, is shown as CMV 181. Within the third expression cassette there may also be a polyadenylation sequence, e.g., a SV40 polyadenylation sequence (not shown). The remaining components are the same as show in FIG. 6, followed by second ITR, labeled as RIR to denote that it is the right independent repeat 192.

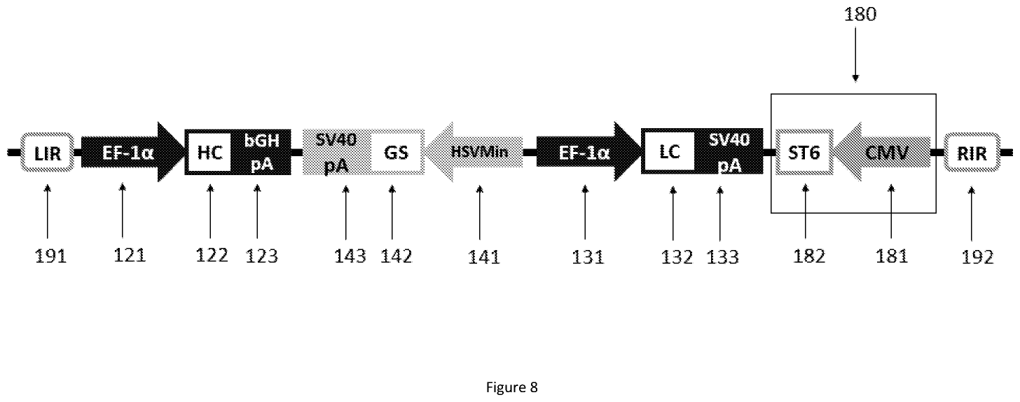

FIG. 8 is similar to FIG. 7 except that the third expression cassette or post-translational modification cassette is located between the second expression cassette, which as shown contains elements 131 (EF-1α promoter), 132 (LC), and 133 (SV40 pA), and the RIR 192. As with FIG. 7, the orientation of the cassettes alternates. However, unlike in FIG. 7, here, with respect to the selection cassette, the third expression cassette is upstream, with the second expression cassette being located between the selection cassette and the third expression cassette.

By way of further example, the third expression cassette or post-translational modification cassette comprises a nucleotide sequence that codes for SEQ ID NO: 37 (ST6Gal1): Met Ile His Thr Asn Leu Lys Lys Lys Phe Ser Tyr Phe Ile Leu Ala Phe Leu Leu Phe Ala Leu Ile Cys Val Trp Lys Lys Gly Ser Tyr Glu Ala Leu Lys Leu Gln Ala Lys Glu Phe Gln Val Thr Arg Ser Leu Glu Lys Leu Ala Met Arg Ser Gly Ser Gln Ser Met Ser Ser Ser Ser Lys Gln Asp Pro Lys Gln Asp Ser Gln Val Leu Ser His Ala Arg Val Thr Ala Lys Val Lys Pro Glu Ala Ser Phe Gln Val Trp Asn Lys Asp Ser Ser Ser Lys Asn Leu Ile Pro Arg Leu Gln Lys Ile Trp Lys Asn Tyr Leu Ser Met Asn Lys Tyr Lys Val Ser Tyr Lys Gly Pro Gly Pro Gly Ile Lys Phe Ser Ala Glu Ala Leu Arg Cys His Leu Arg Asp His Val Asn Val Ser Met Val Glu Val Thr Asp Phe Pro Phe Asn Thr Ser Glu Trp Glu Gly Tyr Leu Pro Lys Glu Ser Ile Arg Thr Lys Ala Gly Pro Trp Gly Arg Cys Ala Val Val Ser Ser Ala Gly Ser Leu Lys Ser Ser Gln Leu Gly Arg Glu Ile Asp Asp His Asp Ala Val Leu Arg Phe Asn Gly Ala Pro Thr Ala Asn Phe Gln Gln Asp Val Gly Thr Lys Thr Thr Ile Arg Leu Met Asn Ser Gln Leu Val Thr Thr Glu Lys Arg Phe Leu Lys Asp Ser Leu Tyr Asn Glu Gly Ile Leu Ile Val Trp Asp Pro Ser Val Tyr His Ser Asp Ile Pro Lys Trp Tyr Gln Asn Pro Asp Tyr Asn Phe Phe Asn Asn Tyr Lys Thr Tyr Arg Lys Leu His Pro Asn Gln Pro Phe Tyr Ile Leu Lys Pro Gln Met Pro Trp Glu Leu Trp Asp Ile Leu Gln Glu Ile Ser Pro Glu Glu Ile Gln Pro Asn Pro Pro Ser Ser Gly Met Leu Gly Ile Ile Ile Met Met Thr Leu Cys Asp Gln Val Asp Ile Tyr Glu Phe Leu Pro Ser Lys Arg Lys Thr Asp Val Cys Tyr Tyr Tyr Gln Lys Phe Phe Asp Ser Ala Cys Thr Met Gly Ala Tyr His Pro Len Len Tyr Glu Lys Asn Len Val Lys His Len Asn Gln Gly Thr Asp Glu Asp Ile Tyr Len Len Gly Lys Ala Thr Len Pro Gly Phe Arg Thr Ile His Cys.

In some embodiments, the third expression cassette or post-translational modification cassette comprises a nucleotide sequence that codes for an amino acid sequence that is at least 80%, at least 85%, at least 90%, at least 95%, at least 98% or 100% identical to SEQ ID NO: 37 over at least 300 consecutive amino acids or over the entire sequence of SEQ ID NO: 37. In some embodiments, the third expression cassette or post-translational modification cassette codes for a polypeptide that comprises, consists essentially of or consists of the amino acids of SEQ ID NO: 37.

In some embodiments, the third expression cassette or post-translational modification cassette comprises a nucleotide sequence that codes for an amino acid sequence that is derived from or is a truncated version of ST6Gal1 and that is between 280 and 350 amino acids long or between 300 and 325 amino acids long.

By way of further example, the third expression cassette or post-translational modification cassette comprises a nucleotide sequence that codes for SEQ ID NO: 38 (ST6Gal2): Met Lys Pro His Len Lys Gln Trp Arg Gln Arg Met Len Phe Gly Ile Phe Ala Trp Gly Len Len Phe Len Len Ile Phe Ile Tyr Phe Thr Asp Ser Asn Pro Ala Glu Pro Val Pro Ser Ser Len Ser Phe Len Glu Thr Arg Arg Len Len Pro Val Gln Gly Lys Gln Arg Ala Ile Met Gly Ala Ala His Glu Pro Ser Pro Pro Gly Gly Len Asp Ala Arg Gln Ala Len Pro Arg Ala His Pro Ala Gly Ser Phe His Ala Gly Pro Gly Asp Len Gln Lys Trp Ala Gln Ser Gln Asp Gly Phe Glu His Lys Glu Phe Phe Ser Ser Gln Val Gly Arg Lys Ser Gln Ser Ala Phe Tyr Pro Glu Asp Asp Asp Tyr Phe Phe Ala Ala Gly Gln Pro Gly Trp His Ser His Thr Gln Gly Thr Len Gly Phe Pro Ser Pro Gly Glu Pro Gly Pro Arg Glu Gly Ala Phe Pro Ala Ala Gln Val Gln Arg Arg Arg Val Lys Lys Arg His Arg Arg Gln Arg Arg Ser His Val Len Glu Glu Gly Asp Asp Gly Asp Arg Len Tyr Ser Ser Met Ser Arg Ala Phe Len Tyr Arg Len Trp Lys Gly Asn Val Ser Ser Lys Met Len Asn Pro Arg Len Gln Lys Ala Met Lys Asp Tyr Len Thr Ala Asn Lys His Gly Val Arg Phe Arg Gly Lys Arg Glu Ala Gly Len Ser Arg Ala Gln Len Len Cys Gln Len Arg Ser Arg Ala Arg Val Arg Thr Len Asp Gly Thr Glu Ala Pro Phe Ser Ala Len Gly Trp Arg Arg Len Val Pro Ala Val Pro Len Ser Gln Len His Pro Arg Gly Len Arg Ser Cys Ala Val Val Met Ser Ala Gly Ala Ile Len Asn Ser Ser Len Gly Glu Glu Ile Asp Ser His Asp Ala Val Len Arg Phe Asn Ser Ala Pro Thr Arg Gly Tyr Glu Lys Asp Val Gly Asn Lys Thr Thr Ile Arg Ile Ile Asn Ser Gln Ile Len Thr Asn Pro Ser His His Phe Ile Asp Ser Ser Len Tyr Lys Asp Val Ile Len Val Ala Trp Asp Pro Ala Pro Tyr Ser Ala Asn Len Asn Len Trp Tyr Lys Lys Pro Asp Tyr Asn Len Phe Thr Pro Tyr Ile Gln His Arg Gln Arg Asn Pro Asn Gln Pro Phe Tyr Ile Len His Pro Lys Phe Ile Trp Gln Len Trp Asp Ile Ile Gln Glu Asn Thr Lys Glu Lys Ile Gln Pro Asn Pro Pro Ser Ser Gly Phe Ile Gly Ile Len Ile Met Met Ser Met Cys Arg Glu Val His Val Tyr Glu Tyr Ile Pro Ser Val Arg Gln Thr Glu Len Cys His Tyr His Glu Len Tyr Tyr Asp Ala Ala Cys Thr Len Gly Ala Tyr His Pro Len Len Tyr Glu Lys Len Len Val Gln Arg Len Asn Met Gly Thr Gln Gly Asp Len His Arg Lys Gly Lys Val Val Len Pro Gly Phe Gln Ala Val His Cys Pro Ala Pro Ser Pro Val Ile Pro His Ser.

In some embodiments, the third expression cassette comprises a nucleotide sequence that codes for an amino acid sequence that is at least 80%, at least 85%, at least 90%, at least 95%, at least 98% or 100% identical to SEQ ID NO: 38 over at least 300 consecutive amino acids or over the entire sequence of SEQ ID NO: 38.

In some embodiments, the third expression cassette or post-translational modification cassette comprises a nucleotide sequence that codes for an amino acid sequence that is derived from or is a truncated version of ST6Gal2 and that is between 300 and 375 amino acids long or between 325 and 350 amino acids long. In some embodiments, the third expression cassette codes for a polypeptide that comprises, consists essentially of or consists of the amino acids of SEQ ID NO: 38.

In some embodiments, the third expression cassette or post-translational modification cassette comprises a nucleotide sequence that codes for SEQ ID NO: 41 (Homo sapiens ST6 beta-galactosamide alpha-2,6-sialyltranferase 1; accession number BC040009.1):

MIHTNLKKKFSCCVLVFLLFAVICVWKEKKKGSYYDSFKLQTKEFQVL
KSLGKLAMGSDSQSVSSSSTQDPHRGRQTLGSLRGLAKAKPEASFQVW
NKDSSSKNLIPRLQKIWKNYLSMNKYKVSYKGPGPGIKFSAEALRCHL
RDHVNVSMVEVTDFPFNTSEWEGYLPKESIRTKAGPWGRCAVVSSAGS
LKSSQLGREIDDHDAVLRFNGAPTANFQQDVGTKTTIRLMNSQLVTTE
KRFLKDSLYNEGILIVWDPSVYHSDIPKWYQNPDYNFFNNYKTYRKLH
PNQPFYILKPQMPWELWDILQEISPEEIQPNPPSSGMLGIIIMMTLCD
QVDIYEFLPSKRKTDVCYYYQKFFDSACTMGAYHPLLYEKNLVKHLNQ
GTDEDIYLLGKATLPGFRTIHC

In some embodiments, the third expression cassette comprises a nucleotide sequence that codes for an amino acid sequence that is at least 80%, at least 85%, at least 90%, at least 95%, at least 98% or 100% identical to SEQ ID NO: 41 over at least 350 consecutive amino acids or over the entire sequence of SEQ ID NO: 41.

In some embodiments, the third expression cassette or post-translational modification cassette comprises a nucleotide sequence that codes for an amino acid sequence that is derived from or is a truncated version of Homo sapiens ST6 beta-galactosamide alpha-2,6-sialyltranferase 1 and that is between 350 and 450 amino acids long or between 375 and 325 amino acids long. In some embodiments, the third expression cassette codes for a polypeptide that comprises, consists essentially of or consists of the amino acids of SEQ ID NO: 41

In some embodiments, the third expression cassette or post-translational modification cassette comprises a polynucleotide sequence that codes for a glycosyltransferase. Examples of glycosyltransferases include, but are not limited to, a sialyltransferase, a galactosyltransferase, a fucosyltransferase, etc. In some embodiments, the third expression cassette or post-translational modification cassette comprises a polynucleotide sequence selected from the group of genes listed in Table 1 of Nguyen, N. T. B., Lin, J., Tay, S. J. et al. Sci Rep 11, 12969 (2021), which is reproduced below as Table 1.

TABLE 1
Polynucleotide sequences coding for a glycosyltransferase.

| Group | Name | Accession No | Gene |
|---|---|---|---|
| Nucleotide sugar | GALE | NM_000403 | UDP-galactose 4-epimerase |
| synthesis | GNE | NM_005476.5 | UDP-N-acetylglucosamine-2 epimerase |
| | | | (Human) |
| | NANS | NM_018946.3 | Sialic acid synthase |
| | NANP | NM_152667.2 | N-acetylneuraminic acid phosphatase |
| | CMAS | NM_018686.5 | Cytidine monophospho-sialic acid synthase |
| | CST | NM_006416.4 | CMP-sialic acid transporter |
| Nucleotide sugar | UGT | NM_005660.2 | UDP-galactose transporter |
| transporter | UGNT | NM_032826.4 | UDP-N-aetylglucosamine transporter |
| | GFT | NM_018389.4 | GDP-fucose transporter |
| | GANC | NM_198141.2 | Neutral α-glucosidase C |
| | MANEA | NM_024641 | Endo-α mannosidase |
| | MANIAI | NM_005907 | Mannosyl-oligosaccharide 1,2-α-mannosidase |
| | | | IA |
| | MANIB | AF-027156 | Mannosyl-oligosaccharide 1,2-α-mannosidase |
| | | | IB |
| Glycan-processing | MANIC1 | AF_261655 | Mannosyl-oligosaccharide 1,2-α-mannosidase |
| glycosidase | | | IC (isoform 1) |
| | MAN2A2 | NM_006122.2 | α-mannosidase, 2A member 2 |
| | MAN2B1 | NM_000528.3 | α-mannosidase, class 2B, member 1 |
| | MAN2B2 | NM_015274.2 | α-mannosidase, class 2B, member 2 |
| | MAN2C1 | NM_006715.3 | α-mannosidase, class 2C, member 1 |
| | MGAT1 | NM_001114618.1 | α-1,3-mannosyl-glycoprotein 2-β-N- |
| | | | acetylglucosaminyltransferase |
| | MGAT2 | BC_006390 | α-1,6-mannosyl-glycoprotein 2-β-N- |
| | | | acetylglucosaminyltransferase |
| | MGAT3 | NM_002409.4 | β-1,4-mannosyl-glycoprotein 4-β-N- |
| | | | acetylglucosaminyltransferase |
| N-Glycan chain | MGAT4A | NM_012214.2 | α-1,3-mannosyl-glycoprotein 4-β-N- |
| extension | | | acetylglucosaminyltransferase A |
| | MGAT4B | AB_000624 | α-1,3-mannosyl-glycoprotein 4-β-N- |
| | | | acetylglucosaminyltransferase B |
| | MGAT4C | BC_064141 | α-1,3-mannosyl-glycoprotein 4-β-N- |
| | | | acetylglucosaminyltransferase C |
| | MGAT5 | NM_002410.4 | α-1,6-mannosyl-glycoprotein 6-β-N- |
| | | | acetylglucosaminyltransferase |
| | MGAT5B | NM_144677.2 | α-1,6-mannosyl-glycoprotein 6-β-N- |
| | | | acetylglucosaminyltransferase B |
| | B4GALT1 | NM_001497.3 | β-1,4-Galactosyltransferase 1 |
| | B4GALT2 | NM_030587.2 | β-1,4-Galactosyltransferase 2 |
| | B4GALT3 | NM_001199873.1 | β-1,4-Galactosyltransferase 3 |
| Galactosylation | B4GALT4 | NM_212543.1 | β-1,4-Galactosyltransferase 4 |
| | B4GALT5 | NM_004776.3 | β-1,4-Galactosyltransferase 5 |
| | B4GALT6 | NM_004775.3 | β-1,4-Galactosyltransferase 6 |
| | B4GALT7 | NM_007255.2 | β-1,4-Galactosyltransferase 7 |
| | ST3GAL1 | NM_174963.3 | β-Galactoside-α-2,3-sialyltransferase 1 |
| | ST3GAL2 | NM_006927.3 | β-Galactoside-α-2,3-sialyltransferase 2 |
| | ST3GAL3 | NM_174963.3 | β-Galactoside-α-2,3-sialyltransferase 3 |
| Sialylation | ST3GAL4 | NM_001254757.1 | β-Galactoside-α-2,3-sialyltransferase 4 |
| | ST3GAL5 | NM_03896.3 | β-Galactoside-α-2,3-sialyltransferase 5 |
| | ST3GAL6 | NM_006100.3 | β-Galactoside-α-2,3-sialyltransferase 6 |
| | ST6GAL1 | BC_0400009 | β-Galactoside-α-2,6-sialyltransferase 1 |
| | ST6GAL2 | NM_032528.2 | β-Galactoside-α-2,6-sialyltransferase 2 |
| Fucosylation | FUT8 | NM_178155.2 | α-1,6-Fucosyltransferase |

FIG. 7 and FIG. 8 are representations of cargo regions that comprise, consist essentially of, or consist of three expression cassettes and one selection cassette. Polynucleotides or vectors that contain these cargo regions may be simultaneously or sequentially transfected into a cell with an additional vector that codes for a transposase protein or transfected into a cell that already contains the transposase protein or a vector capable of expressing the transposase protein. Thus, in some embodiments, there is a kit that comprises: (1) a first vector that comprises each of a first expression cassette, a second expression cassette, a third expression cassette and a selection cassette as described in various embodiments of the present disclosure; and (2) a second vector that encodes for a transposase, wherein the transposase is capable of causing the first expression cassette, the second expression cassette, the third expression cassette and the selection cassette to being integrated into a host genome. In some embodiments, at least one or two of the first expression cassette, the second expression cassette, and the third expression cassette codes for an antibody or a component of an antibody and at least one of the first expression cassette, the second expression cassette, and the third expression cassette codes for an enzyme or the catalytic region of an enzyme that is capable of causing a post-translational modification of the antibody or component thereof.

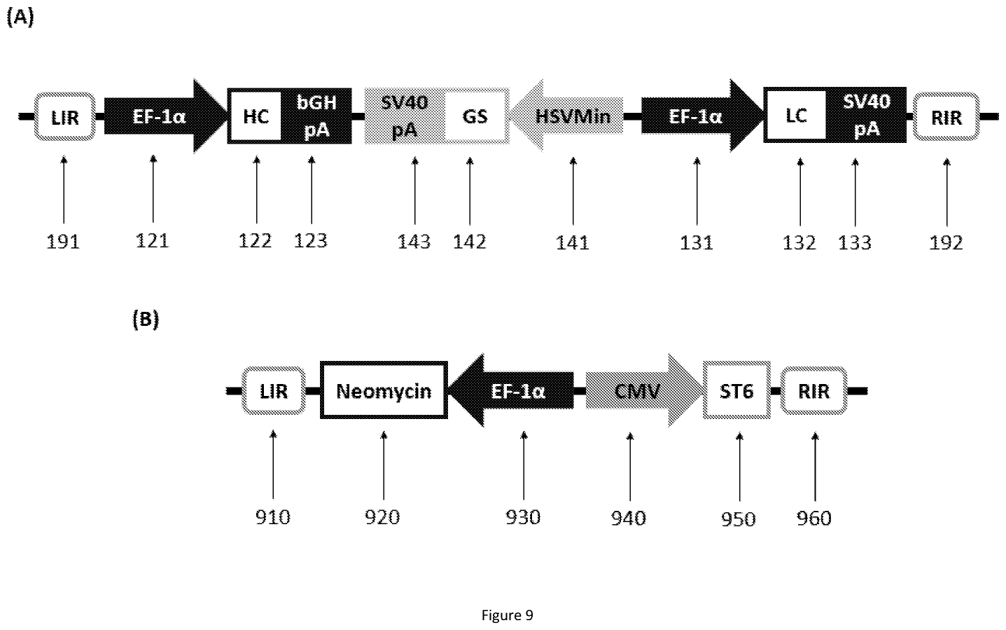

In contrast to the embodiments represented in FIGS. 7 and 8, FIGS. 9A and 9B represent an embodiment in which the first expression cassette (containing elements 121, 122, and 123), the second expression cassette (containing elements 131, 132, and 133), and the selection cassette (containing elements 143, 142, and 141), are located between the LIR 191 and the RIR 192 in a polynucleotide that may be part of a first vector, while a second polynucleotide that may be part of a second vector codes for the post-translation protein, e.g., ST6Gal1 or ST6Gal2 or a fragment or derivative thereof (e.g., SEQ ID NO: 37 or SEQ ID NO: 38 or a sequence that comprises at least 80%, at least 85%, at least 90%, at least 95%, or at least 98% sequence identity to SEQ ID NO: 37 or SEQ ID NO: 38 over at least 300 amino acids). FIG. 9B shows its LIR, 910, selection marker e.g., neomycin 920, a fourth promoter e.g., EF-1α 930, a fifth promoter, 940, and a polynucleotide sequence that codes for a post-translation protein 950 followed by the RIR 960. In this second polynucleotide, which may be a vector, the selection marker, which e.g., may be an antibiotic selection marker, as well as its promoter, may be oriented in the same (not shown) or opposite direction as that of the region that codes for the post-translation modification protein and its marker. These two polynucleotides or vectors may be part of a kit and may be introduced simultaneously or sequentially into a cell. Further, although not shown, between elements 950 and 960, i.e., downstream of the sequence that codes for the post-translation protein, there may be a polyadenylation sequence. Optionally, a third vector that code for a transposase may be introduced simultaneously with one or both of the aforementioned vectors or sequentially with those vectors. Alternatively, the first vector and the second vector may be transfected into a cell that already contains the transposase protein or a vector capable of expressing the transposase protein.

When there is a second vector that codes for a post-translational protein, in some embodiments, that vector may be transposable by the same transposase as the first vector. In other embodiments, it may be transposable by a different transposase. In still other embodiments, it may be integrated into the host genome by another method that is now known or that comes to be known and that a person of ordinary skill in the art would appreciate as being of use in connection with the present disclosure or it may be expressible in a cell without integration into a host's genome.

### Kits for Generating and Modifying a Protein

The present disclosure also provides kits for the generation and modification of one or more polypeptides. Kits that are within the scope of the present disclosure are kits that comprise: (1) a first polynucleotide that comprises a cloning cassette or an expression cassette that codes for a polypeptide, and a second polynucleotide that comprises a post-translational modification cassette that codes for a post-translational modification protein, wherein the post-translational modification protein is capable of modifying the polypeptide; or (2) a first polynucleotide that comprises a first cloning cassette or expression cassette that codes for a first polypeptide, and a second polynucleotide that comprises both a post-translational modification cassette that codes for a post-translational protein, and a second cloning cassette or expression cassette that codes for a second polypeptide, wherein the post-translational modification protein is capable of modifying either or both of the first polypeptide and the second polypeptide; or (3) a first polynucleotide that comprises a first cloning cassette or expression cassette that codes for a first polypeptide, a second polynucleotide that comprises a second cloning cassette or expression cassette that codes for a second polypeptide, and a third polynucleotide that codes for a post-translational protein, wherein the post-translational protein is capable of modifying either or both of the first polypeptide and the second polypeptide; or (4) a first polynucleotide that comprises a first cloning cassette or expression cassette that codes for a first polypeptide and a second cloning cassette or expression cassette that codes for a second polypeptide; and a second polynucleotide that codes for a post-translational protein, wherein the post-translational protein is capable of modifying either or both of the first polypeptide and the second polypeptide. In some embodiments, the kit of the present disclosure comprises one or more polynucleotides comprising 1, 2, 3, 4, 5, 6, 7, 8 or more cloning cassettes or expression cassettes coding for 1, 2, 3, 4, 5, 6, 7, 8 or more polypeptides. In some embodiments, the kit of the present disclosure comprises one or more polynucleotides comprising 1, 2, 3, 4, 5, 6, 7, 8 or more cloning cassettes or expression cassettes coding for 1, 2, 3, 4, 5, 6, 7, 8 or more polypeptides, and another polynucleotide that codes for a post-translational protein, wherein the post-translational protein is capable of modifying the 1, 2, 3, 4, 5, 6, 7, 8 or more polypeptides. Optionally, all kits of present disclosure may comprise another polynucleotide that comprises an expression cassette that codes for a polypeptide as expression control. Optionally, any or all of the aforementioned cassettes may comprise promoters and polyadenylations sequences that are described elsewhere in this disclosure.

### Genetic Delivery Systems

The present disclosure also provides genetic delivery systems. In these systems, in addition to the polynucleotide of the present disclosure, optionally any additional elements for effective integration of the polynucleotide into a host DNA or any additional polynucleotides such as plasmids or mRNA that code for these additional elements may be included.

When the polynucleotide is a transposon vector, a transposase or a vector such as a plasmid or an mRNA that encodes the transposase may be provided.

Examples of transposases that can be used in the genetic delivery systems of the present disclosure include, but are not limited to, (i) a Sleeping Beauty transposase (for example as disclosed in U.S. Pat. No. 9,228,180), such as a hyperactive Sleeping Beauty (SB100X) transposase or SB10 transposase; (ii) a Helitron transposase (for example, as disclosed in WO 2019/173636); (iii) a Tol2 transposase (for example, as disclosed in WO 2019/173636) or (iv) a TcBuster transposase or a hyperactive TcBuster transposase (for example, as disclosed in WO 2019/173636).

In some embodiments, the transposases that may be used in connection with the embodiments of the present disclosure in which the polynucleotides are transposons, are transposases that are now known or that come to be known and may, for example, be used in Tc1/mariner systems.

Examples of the Sleeping Beauty transposases include, but are not limited to, SB10 or SB100X, wherein SB100X comprises the following sequence:

(SEQ ID NO: 42)
MGKSKEISQDLRKRIVDLHKSGSSLGAISKRLAVPRSSVQTIVRKYKH
HGTTQPSYRSGRRRVLSPRDERTLVRKVQINPRTTAKDLVKMLEETGT
KVSISTVKRVLYRHNLKGHSARKKPLLQNRHKKARLRFATAHGDKDRT
FWRNVLWSDETKIELFGHNDHRYVWRKKGEACKPKNTIPTVKHGGGSI
MLWGCFAAGGTGALHKIDGIMDAVQYVDILKQHLKTSVRKLKLGRKWV
FQHDNDPKHTSKVVAKWLKDNKVKVLEWPSQSPDLNPIENLWAELKKR
VRARRPTNLTQLHQLCQEEWAKIHPNYCGKLVEGYPKRLTQVKQFKGN
ATKY

as well as derivatives of the Tc1/mariner systems, e.g., Frog Prince, HsmarI, and Minos, which have the sequences noted in Table 2 below.

TABLE 2

| Transposase | Amino sequence | Source |
|---|---|---|
| Frog Prince | MPRPKEIQEQLRKKVIEIY | US20050241007A1 |
| | QSGKGYKAISKALGIQRT | (SEQ ID NO: 34) |
| | TVRAIIHKWRRHGTVVNL | |
| | PRSGRPPKITPRAQRRLIQ | |
| | EVTKDPTTTSKELQASLAS | |
| | VKVSVHASTIRKRLGKNG | |
| | LHGRVPRRKPLLSKKNIK | |
| | ARLNFSTTHLDDPQDFWD | |
| | NILWTDETKVELFGRCVS | |
| | KYIWRRRNTAFHKKNIIPT | |
| | VKYGGGSVMVWGCFAAS | |
| | GPGRLAVIKGTMNSAVYQ | |
| | EILKENVRPSVRVLKLKRT | |
| | WVLQQDNDPKHTSKSTTE | |
| | WLKKNKMKTLEWPSQSP | |
| | DLNPIEMLWYDLKKAVH | |
| | ARKPSNVTELGQFCKDEW | |
| | AKIPPGRCKSLIARYRKRL | |
| | VAVVAAKGGPTSY | |
| Minos | MVRGKPISKEIRVLIRDYF | https://www. |
| | KSGKTLTEISKQLNLPKSS | uniprot.org/ |
| | VHGVIQIFKKNGNIENNIA | uniprot/A8E1U2 |
| | NRGRTSAITPRDKRQLAKI | (SEQ ID NO: 35) |
| | VKADRRQSLRNLASKWS | |
| | QTIGKTVKREWTRQQLKS | |
| | IGYGFYKAKEKPLLTLRQ | |
| | KKKRLQWARERMSWTQR | |
| | QWDTIIFSDEAKFDVSVG | |
| | DTRKRVIRKRSETYHKDC | |
| | LKRTTKFPASTMVWGCM | |
| | SAKGLGKLHFIEGTVNAE | |
| | KYINILQDSLLPSIPKLSDC | |
| | GEFTFQQDGASSHTAKRT | |
| | KNWLQYNQMEVLDWPS | |
| | NSPDLSPIENIWWLMKNQ | |
| | LRNEPQRNISDLKIKLQEM | |
| | WDSISQEHCKNLLSSMPK | |
| | RVKCVMQAKGDVTQF | |
| HsmarI | MEMMLDKKQIRAIFLFEF | WO2006108525A1 |
| | KMGRKAAETTRNINNAFG | (SEQ ID NO: 36) |
| | PGTANERTVQWWFKKFR | |
| | KGDESLEDEERSGRPSEV | |
| | DNDQLRAIIEADPLTTTRE | |
| | VAEELNVDHSTVVRHLK | |
| | QIGKVKKLDKWVPHELSE | |
| | NQKNRRFEVSSSLLLRNN | |
| | NEPFLDRIVTCDEKWILYD | |
| | NRRRSAQWLDREEAPKHF | |
| | PKPNLHQKKVMVTVWWS | |
| | AAGVIHYSFLNPGETITSE | |
| | KYCQQIDEMHRKLQRLQP | |
| | ALVNRKGPILLHDNARPH | |
| | VAQPTLQKLNELGYEVLP | |
| | HPPYSPDLSPTDYHFFKHL | |
| | DNFLQGKRFHNQQDAEN | |
| | AFQEFVESRSTDFYATGIN | |
| | KLISRWQKCVDCNGSYFD | |

Also included within the scope of this disclosure are transposases that are at least 80% similar to, at least 85% similar to (i.e., have that degree of sequence identity with), at least 90% similar to, and at least 95% similar to any of the aforementioned transposases. Similarly, within the scope of this disclosure are polynucleotides, including but not limited to vectors that comprise sequences that code for or are at least 80% similar to, at least 85% similar to, at least 90% similar to, and at least 95% similar to a polynucleotide sequence that codes for any of the aforementioned transposases.

### Method of Introduction of Exogenous Nucleotide Sequences

The present disclosure also provides methods for introducing an exogenous polynucleotide sequence into a host cell. The present disclosure also provides methods for introducing an exogenous polynucleotide sequence into a nucleotide sequence in a host cell. The present disclosure also provides methods for introducing and integrating an exogenous polynucleotide sequence into a nucleotide sequence in a host cell. This may be done in vitro, in vivo, or ex vivo. In some embodiments, the method for introducing an exogenous polynucleotide into a nucleotide sequence in a host cell comprises introducing a viral vector in the cell, such as a lentiviral vector or a retroviral vector. In other embodiments, the method for introducing an exogenous polynucleotide into a target nucleotide sequence in a host cell comprises introducing a transposon vector in the cell. The cell may already contain a transposase or a vector that is capable of expressing the transposase. Alternatively, a transposase or vector or mRNA that is capable of expressing the transposase may be introduced into the cell simultaneously with the transposon or before or after introduction of the transposon containing vector. Further, the integration of the exogenous polynucleotide may be to chromosomal DNA or non-chromosomal DNA. In another embodiment, the integration of the exogenous polynucleotide may be to nuclear DNA or extra-nuclear DNA, such as mitochondrial DNA.

In some embodiments, the method of integration of the polynucleotide of the present disclosure is performed under conditions that allow for a random integration. Conditions that allow for a random integration include, but are not limited to, the use of a polynucleotide that does not comprise ITRs repeat sequences or the use of a cell not containing a transposase.

In some embodiments, the method of integration of the polynucleotide of the present disclosure occurs through more targeted systems for integration such as systems that make use of transposon technologies. If the cell is a eukaryotic cell, the transposon may, in some embodiments, be delivered to the nucleus of the cell.

After a polynucleotide of the present disclosure is introduced into a cell, the cell may be deemed to be a modified cell regardless of whether the cargo region has been integrated, i.e., cut and paste and thus integrated into the host cell's DNA. In some embodiments, the polynucleotide of the present disclosure is not integrated into the host cell's DNA and it may be maintained as an episome.

The cells may, for example, be any host cells that may be cultured under selection media and that can be selected by the selection cassette. In some embodiments, the host cell is a vertebrate cell. In some embodiments, the cells are mammalian cells. Examples of host cells include, but are not limited, to human cells, rodent cells, or avian cells. In some embodiments, the host cell is selected from the group consisting of Chinese Hamster Ovary (CHO), EB66, NSO murine myeloma, PER.C6, Baby hamster kidney, Human Embryonic Kidney derived (such as HEK293 cell lines, HEK293T, HEK293F or HEK293N3S), Chicken embryo fibroblast, Madin Darby bovine kidney, Madin Darby canine kidney, and VERO cells. In some embodiments, the cell is a Chinese Hamster Ovary cell line selected from the group consisting of a CHO-K1, CHO—S and CHO DG44 progenitor cell and modified versions thereof. In some embodiments, the host cell is a modified cell, such as a gene/s knock-out cell. In some embodiments, the host cell is a GS KO cell line if the selection cassette expresses glutamine synthetase. In some embodiments, the host cell is an ADCC+ cell line if the selection cassette expresses glutamine synthetase.

Because the polynucleotides may be introduced through a vector, known technologies for introducing vectors into cells may be employed. See, for example, Kyung Kim et al. Anal Bioanal Chem. 2010; 397(8): 3173-3178. These technologies include, but are not limited to, gene guns, lipofectamine, lipofectin, electroporation, injection, and complex formation with liposomes or polyethylenimine.

Further, methods for the creation of polynucleotides and vectors such as plasmids and transposons are well known to persons of ordinary skill in the art and include, but are not limited to, restriction enzyme cloning, Gibson assembly (Gibson et al., 2009, PMID: 19363495 DOI: 10.1038/nmeth.1318), Golden Gate cloning (Engler et al., 2008, 10.1371/journal.pone.0003647).

### Applications

Biopharmaceutical drug discovery is reliant on the recombinant expression of proteinogenic and non-proteinogenic biological material in mammalian cell-based manufacturing platforms. The generation of these stably expressing host cells requires screening methodologies, because cells that are constructed may have a wide range of expression, growth, and stability characteristics. The polynucleotides and modified cells of the present disclosure may be used to reduce the burden of obtaining a commercially viable production host cell when clones need to be screened and may be used in applications that are now known or that come to be known. Among these applications are bioproduction methods, e.g., developing biopharmaceuticals or non-protein biotherapeutics and utilizing a modified cell of the present disclosure to generate products for the biotechnology and pharmaceutical industry.

Additional applications of the present disclosure, include, but are not limited to, cell therapy, gene therapy, the generation of transgenic cells lines, the creation of induced pluripotent stem cell (iPSC) through reprogramming, phenotypic-driven insertional mutagenesis screens, and germline gene transfer. Other suitable stem cells that can be generated by the methods of the present disclosure include, but are not limited to, mammalian cells such as human stem cells, including hematopoietic, neural, embryonic, mesenchymal, mesodermal, liver, pancreatic, muscle, and retinal stem cells. In one embodiment, the methods of the present disclosure may be used to generate therapeutic cells such as CAR-T cells.

For cell and gene therapy, in one embodiment the present disclosure provides a method of introducing a nucleic acid or gene of interest into a subject in need of that nucleic acid or gene of interest. Suitable subjects may comprise or be any eukaryotic cell such as a plant, mammalian, human cell, etc. Where a subject, such as a human patient, has a pathology associated with a loss of function mutation, gene therapy has the potential to restore health. Gene therapy involves introducing an expression construct into the cell of a patient. This can be performed ex vivo or in vivo. Accordingly, the polynucleotides of the present disclosure may be used in systems and methods that are used to generate engineered cells that, once re-introduced into the patient, can achieve restoration of a missing function. In addition, the introduction of a therapeutic antibody may be beneficial in situations where cellular signaling is disrupted. Further, engineered cells may be used to secrete a therapeutic antibody at appropriate locations at high levels in a patient. The present disclosure also provides a system enabling the introduction of a gene of interest to a subject such as a human patient and therefore provides methods for treating disease and pharmaceutical compositions, e.g., medicaments for use in treating diseases, disorders, or conditions.

Another application of the present disclosure includes the generation of a cell line that can be used to generate reference standards. Thus, the present disclosure provides a method to generate a cell line comprising multiple copies of an endogenous gene of interest or a non-endogenous gene of interest. Examples of genes that may be useful as reference standards include, but are not limited to, ERBB/Her2, MET, CDK4 and CD274/PD-L1. For generating reference standards, a method in accordance with the present disclosure may, e.g., comprise selecting clones with known copy numbers and/or generating a cell in such a way that a defined copy number is obtained. Cell lines that may be used for generating reference standards include, but are not limited to, CHO cells, HAP1 and eHAP cell lines.

By way of non-limiting examples, the modified cells (e.g., CHO cells) of the present disclosure can be used for the following applications: generation of biological material such as monoclonal antibodies, Fc-fusion proteins, multi-specific antibodies or proteins (bi-specific proteins, tri-specific proteins) or any other therapeutic proteins; and development of biopharmaceuticals and difficult to express proteins and other non-protein biotherapeutics, such as viral vectors. The modified cells of the present disclosure can also be used in bioproduction manufacturing methods using perfusion systems, such as continuous manufacturing and intensified fed-batch.

Other applications of the transposon system of the present disclosure include a transposon system or method comprising a copy and paste transposon as described herein for the use as a tool for mutagenesis techniques.

### EXAMPLES

For Examples 1-6, transposon vectors comprising the polynucleotides of the present disclosure were co-transfected with transposase mRNA into GS KO cells via electroporation. The cells were transfected with a transposon vector expressing trastuzumab antibody and comprising different promoter sequences controlling the expression of the selection cassette glutamine synthetase. The promoters' details and sequences tested are depicted in Table 3 below. Stably expressing pools were generated through selection in glutamine free media, which was initiated 48 h after transfection. Selection was completed and pools were expanded to E125 flasks when cell density had increased on two consecutive counts and was above 0.5×10⁶ and viability had increased between two consecutive counts and was above 70%. Non-transfected cells were used as a control for the selection process. Expressing pools were cultured for 14 days under fed batch overgrow conditions.

### Example 1: Growth Profile of Expressing Pools During Selection

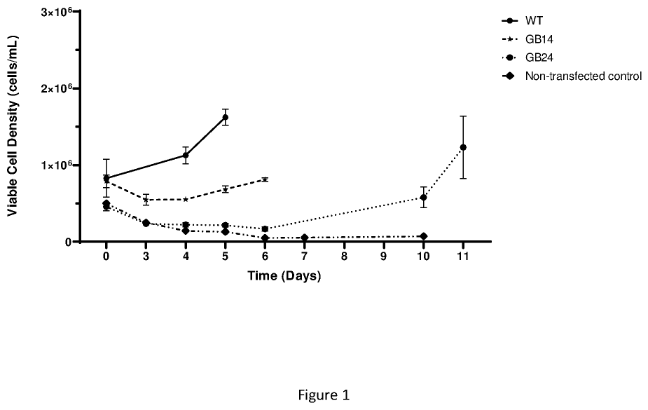

FIG. 1 represents the VCD of the cells transfected with vectors expressing trastuzumab antibody comprising the following promoter sequences controlling the expression of the selection cassette: WT HSVmin promoter fragment sequence WT (SEQ ID NO: 1), HSVmin promoter sequence GB14 (SEQ ID NO: 14), and HSVmin promoter sequence GB24 (SEQ ID NO: 24). See Table 3 below. Selection was initiated 48 h after transfection using glutamine free media. The growth profile was monitored every day or every other day from day 3 of selection. Each line represents the average of three expressing pools generated for each construct: WT promoter, GB14 promoter, GB24 promoter and non-transfected control cells. Error bars represent standard deviation.

The results show that cells transfected with the vector containing the HSVmin promoter WT sequence reached VCD above 0.5×10⁶ cells/ml and viability above 70% on day 5, whereas cells transfected with a vector containing GB14 and GB24 HSVmin promoters reached VCD above 0.5×10⁶ cells/ml and a viability above 70% on day 6 and 11, respectively. Altogether, these results indicate that the selection process for the cells transfected with a vector containing GB14 or GB24 promoters was more stringent than for the cells transfected with the vector carrying the WT sequence of the HSVmin promoter. A stringent selection process can lead to the selection of cells that have integrated a higher number of copies of the selection cassette and, therefore, a higher number of copies of the expression cassette from the vector.

### Example 2: Cell Viability of Expressing Pools During Selection

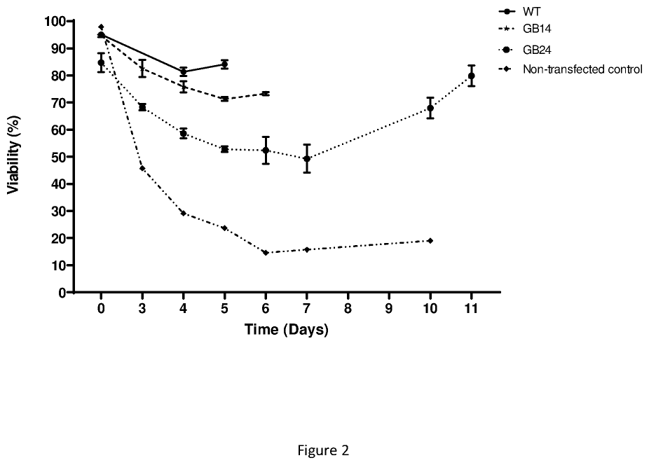

FIG. 2 represents the cell viability of the previous cells transfected with vectors comprising the following HSVmin promoter sequences: WT (SEQ ID NO: 1), GB14 (SEQ ID NO: 14), and GB24 (SEQ ID NO: 24). Selection in glutamine free media was initiated 48 h after transfection. Viability was monitored every day or every other day from day 3 of selection. Selection was complete and pools were expanded to E125 flasks when cell viability was increasing on consecutive measurements and was >70%. Non-transfected cells were used as a control for the selection process. Each line represents the average of three expressing pools generated for each construct. Error bars represent standard deviation.

The data show that cells transfected with the vector containing the HSVmin promoter WT sequence maintained high cell viability throughout the selection process (above 80%). In contrast, the viability of the cells transfected with a vector carrying GB14 or GB24 promoter sequence dropped to 70% and 50% throughout the selection process, respectively. The cell cultures transfected with the vectors carrying GB14 or GB24 promoters were progressed to shake flasks culture one or six days later when compared to the cells transfected with the vector containing the WT promoter sequence. Altogether, these results indicate that the selection process for the cells transfected with a vector containing GB14 or GB24 promoters was more stringent than the selection process for the cells transfected with the vector carrying the WT sequence. A stringent selection process can lead to the selection of cells that have integrated a higher number of copies of the selection cassette and therefore the other expression cassettes on the transposon.

### Example 3: GS Gene Copy Number Assessment

The number of the selection marker Glutamine Synthetase (GS) gene copies integrated into the host cell DNA was determined using droplet digital PCR (ddPCR). Selection in glutamine free media was initiated 48 h after transfection. Cell pellets were collected for each pool after recovery from selection and used for genomic DNA extraction. GS gene specific primers and probes were designed for the ddPCR and 32 Microglobulin was used as the reference gene.

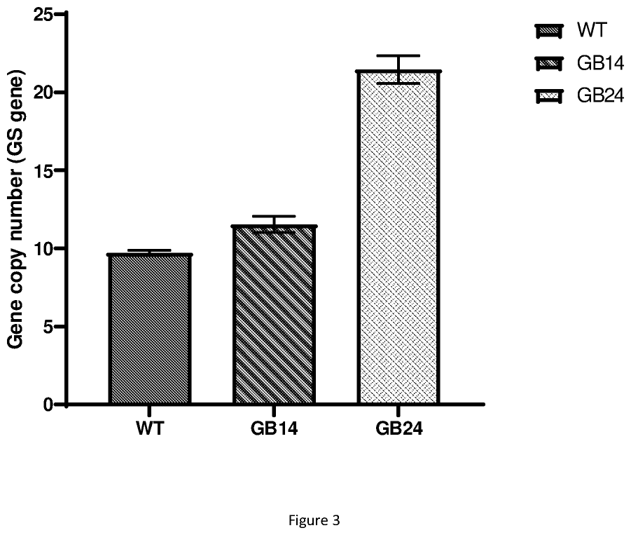

FIG. 3 is a graph that summarizes the number of GS gene copies integrated into the host cell DNA. Each sample was analysed in triplicate and error bars represent standard deviation

The GS copy number analysis shows that more GS cassettes have been integrated into the CHO cells host genome when cells have been transfected with vectors carrying GB14 or GB24 HSVmin promoter sequence, when compared to the cells transfected with the vector containing the HSVmin promoter WT sequence. The increase observed was of 1.2- and 2.2-fold for GB14 and GB24, respectively. These results indicate that, in order to survive the selection process, the cells transfected with vectors carrying GB14 or GB24 promoters need to integrate a higher number of copies of the selection cassette and, therefore, integrate higher number of copies of the transposon's cargo, which could lead to higher expression of the genes of interest (expression cassette).

### Example 4: Pool Productivity Assessment

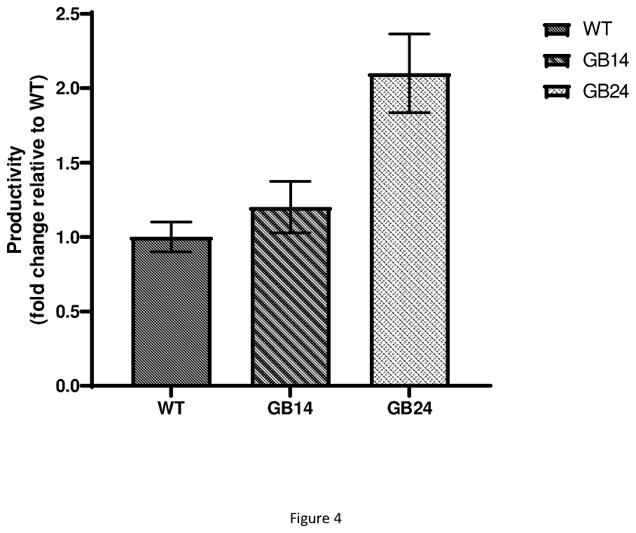

To assess productivity, pools generated with expression vectors comprising WT HSVmin promoter (SEQ ID NO: 1), GB14 HSVmin promoter (SEQ ID NO: 14), and GB24 HSVmin promoter (SEQ ID NO: 24) sequences were enrolled in a fed batch overgrow experiment. The titer was analyzed on day 14 at harvest using an Octet® Protein A Biosensors. Productivity was normalized relative to the WT construct. The results are provided in FIG. 4, in which each bar represents the average of three expressing pools generated for each construct. The samples were analyzed in duplicate and error bars represent standard deviation.

FIG. 4 represents the titer obtained for cells transfected with vectors containing GB14 and GB24 promoter sequences relative to the titer obtained when cells were transfected with a vector containing the WT promoter sequence. It shows an unexpected 1.2 and a 2.1 fold increase in productivity for cells transfected with vectors containing GB14 and GB24 sequences, respectively, compared to cells transfected with the HSVmin WT promoter. These results show that there is a significant advantage in using expression vectors comprising the GB14 or GB24 promoter sequences controlling the expression of a selection cassette, as the pools generated with these vectors show an increase in productivity in relation to the pools transfected with the vector containing the HSVmin promoter WT sequence.

### Example 5: Pool Gene Copy Number and Productivity Assessment

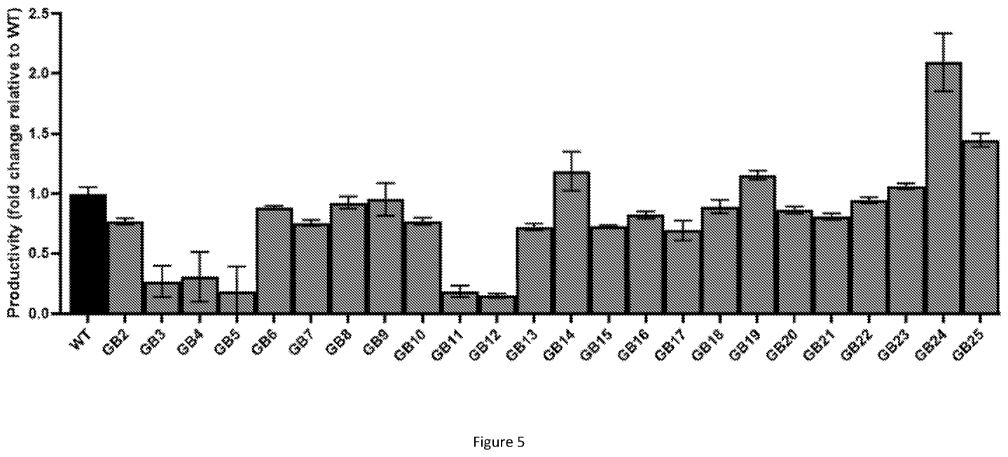

Twenty-five different HSVmin promoter sequences (see sequences SEQ ID NO: 1 to SEQ ID NO: 25 in Table 3) including WT sequence were cloned into the transposon vector to control the expression of the selection cassette and transfected in the CHO-K1 GS KO cells lines. Following selection with Glutamine Synthetase, the recovered pools were incorporated in a fed-batch overgrow experiment. The titer was assessed on day 14 at harvest, using Octet® Protein A Biosensor. The results are provided in FIG. 5 and FIG. 16 in which each bar represents the average of three expressing pools generated for each construct. The samples were analyzed in duplicate and error bars represent standard deviation.

Table 3 contains the Sequence ID, the nucleotide sequence of all the sequences tested, the day the cells transfected with the corresponding vector recovered from selection (“Recovery Day”), and the number of integrated copies of the GS selection cassette: copy number variation (“CNV”).

Interestingly, as shown in FIG. 5, the analysis of the productivity obtained in the different expressing pools revealed that two of the vectors tested out of the twenty-five outperformed the productivity obtained when cells were transfected with the vector carrying the WT sequence. These two vectors are the vectors carrying the promoter sequence set forth in SEQ ID NO: 14 and SEQ ID NO: 24. Summary data is provided in Table 4 below.

As shown in FIG. 16, the analysis of the Glutamine Synthetase copy number revealed that the use of different transposon vectors comprising different HSVMin promoters results in the generation of pools with a wide range of Glutamine Synthetase (GS) copies integrated into the host cell DNA, varying from the integration of 1 copy of the selection cassette with the vector comprising the GB5 promoter to the integration of more than 10 copies with the vector comprising the GB14 promoter and up to the integration of over 20 copies with the vector comprising the GB24 promoter.

### Example 6: Clone Stability Testing

Trastuzumab (Ttz)-expressing pools generated with transposon vectors comprising GB14 or GB24 HSVMin promoter were used for the isolation of clones using a limiting dilution method. A total of 400 clones were isolated from Ttz expressing pools, screened and ranked based on titer for vectors comprising the GB14 or GB24 promoter sequences (200 clones/vector). The 30 clones with highest productivity (15 clones/vector) were cultured for a total of 90 generations. After 90 population doublings, the GS copy number and Ttz expression profile was assessed using ddPCR. For each clone, productivity was assessed at generation 0 (Gen 0) and at generation 90 (Gen 90) through a 14-day fed-batch overgrowth experiment. The titer was analyzed on day 14 at harvest using Octet® Protein A Biosensors.

FIG. 17 shows the copy number assessment (A) and productivity profile (B) of Ttz expressing clones after 90 generations. The results show that all clones generated with transposon vectors comprising GB14 or GB24 promoters maintained the number of GS copies integrated in the host cell genome consistent throughout the 90 generations, demonstrating the genetic stability of the integration into the host genome. Productivity analysis reveals that 29 out of 30 clones stably express Ttz because the titer (productivity) variation between generation 0 and 90 is below 30%. These data indicate that all clones have a desired high stability, as both the GS copy number integration and the productivity levels are maintained throughout 90 generations.

### Example 7: Pool Productivity Assessment in a Different CHO Cell Line Host

Transposon vectors comprising the polynucleotides of the present disclosure (including trastuzumab antibody as the expression cassette and glutamine synthetase as the selection cassette) were co-transfected with transposase mRNA into the GS KO cell line and the ADCC+ cell line hosts via electroporation. The ADCC+ cell line eliminates the cell's natural fucosylation activity to increase therapeutic antibody efficacy and potency. Stably expressing pools were generated in glutamine free media, which was initiated 48 h after transfection. 3 pools each of GS KO-WT promoter, GS KO-GB24 promoter and ADCC+-GB24 promoter were generated. Non-transfected GS KO and ADCC+ cells were used to generate one pool each of non-transfected controls for the selection process. Pools were monitored for their growth profile every day or every other day. On day 5, cells were counted, centrifuged, and resuspended in fresh selection medium. Selection was completed and pools were expanded to E125 flasks when cell density had increased on two consecutive counts and was above 0.5×10⁶ and viability had increased between two consecutive counts and was above 70%. Non-transfected cells were used as a control for the selection process.

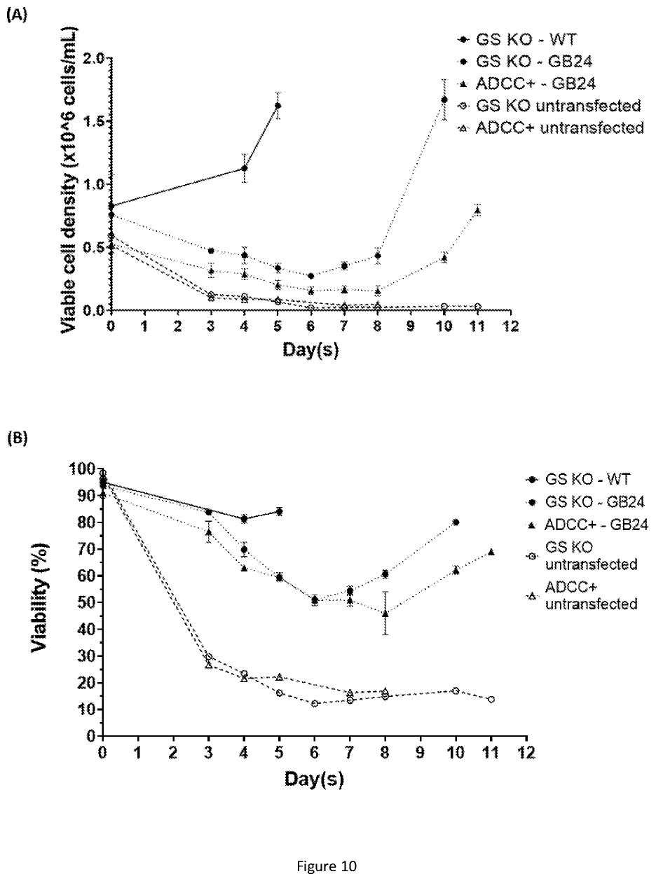

FIG. 10 shows the growth profile of transfected and recovering pools in selection medium whereby (A) represents the viable cell density and (B) represents the percentage viability. The results show that the ADCC+ cell line transfected with a vector containing the HSVMin promoter GB24 sequence reached a VCD above 0.5×10⁶ cells/ml and viability above 70% between day 10 and day 1. This result confirms that the selection process for different cell line hosts transfected with a vector containing GB24 promoter was more stringent than for the GS KO cells transfected with the vector carrying the WT sequence of the HSVmin promoter.

Following recovery from selection, cell pellets were collected for each pool GS KO-WT, GS KO-GB24 and ADCC+-GB24 and the number of Glutamine Synthetase (GS) gene copies integrated in the genome was determined using ddPCR. GS gene specific primers and probes were designed for the ddPCR. B2M was used as the reference gene for the assay.

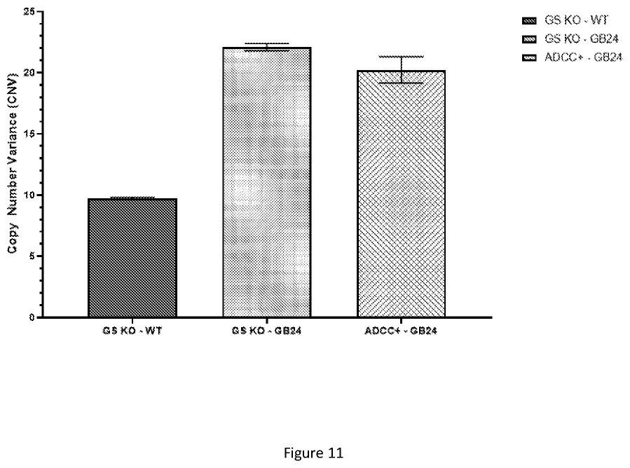

FIG. 11 shows the GS gene copy number variation as observed in the pools recovered from selection. The analysis shows that a comparable number of GS cassettes was integrated into GS KO-GB24 cell line and ADCC+-GB24 cell line. These results confirm that to survive the selection process, the different cell line host ADCC+ transfected with vectors comprising the GB24 promoter, similar to GS KO-GB24, needs to integrate a higher number of copies of the selection cassette and, therefore, integrate higher number of copies of the transposon's cargo, which could lead to higher expression of the genes of interest (cargo expression cassette).

To assess productivity, stably expressing pools GS KO-WT, GS KO-GB24 and ADCC+-GB24 were enrolled in a fed-batch overgrow experiment. This assay was conducted in tubespin bioreactors with a start volume of 10 mL. Cell counts were performed on days 0, 4, 7, 10 and 12 on Vi-CELL™ cell analyzer and glucose was measured using a glucose meter. Pools were fed with HyClone Cell Boost™ 7A (5%) and 7B (0.5%) and glucose on days 4, 7, 10 and 12. Tubespins were incubated at 37° C., 5% CO2 and 225 rpm. Temperature shift to 32° C. was performed on day 5. Titre was analysed using Octet® Protein A Biosensor. Productivity was normalised relative to WT construct.

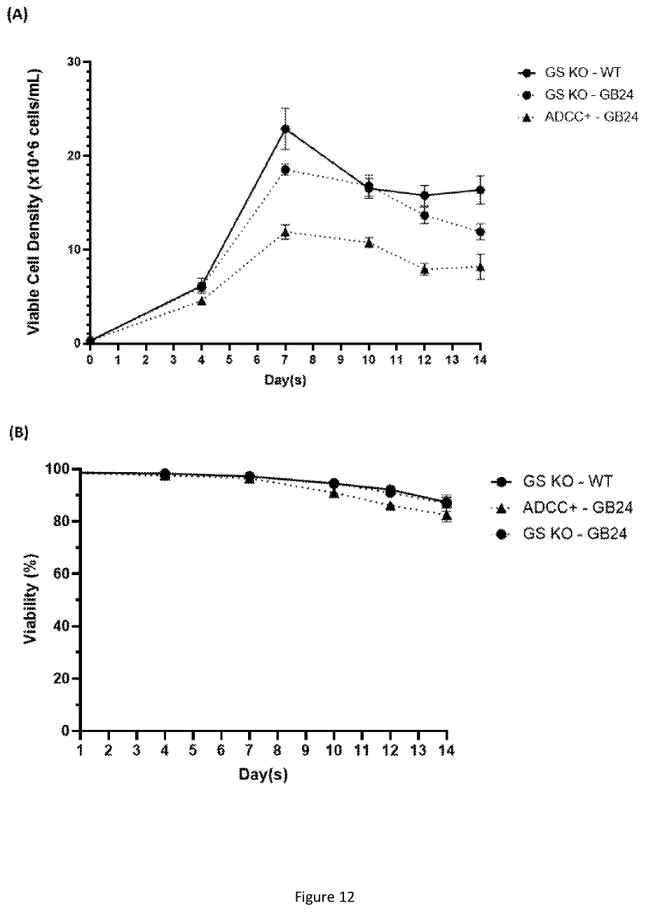

FIG. 12 shows the growth profile of GS KO-WT, GS KO-GB24 and ADCC+-GB24 during the 14-day fed batch culture, whereby (A) represents the viable cell density (VCD) of the pools and (B) represents the percentage viability. The results indicate that the different cell line hosts transfected with vectors carrying GB24 promoter present a growth profile which corresponds to the cell's genotype and high viability throughout the fed-batch process.

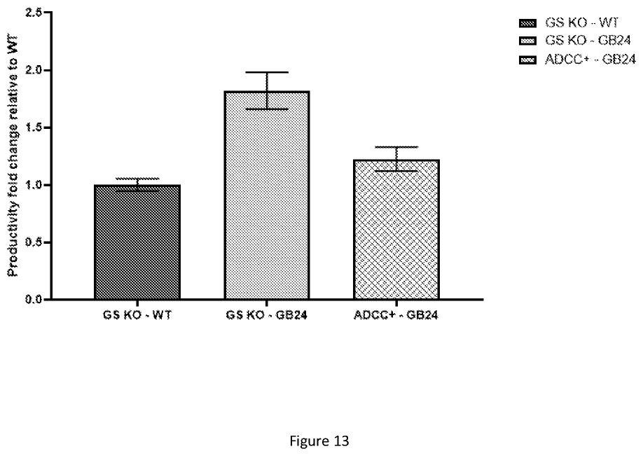

FIG. 13 shows the productivity profile of GS KO-WT, GS KO-GB24 and ADCC+-GB24 during the 14-day fed-batch overgrowth culture. The results show a 1.8-fold and 1.2-fold increase in productivity for GS KO-GB24 and ADCC+-GB24, respectively, compared to cells transfected with the HSVmin WT promoter. The results indicate that that there is a significant advantage in using expression vectors comprising GB24 promoter sequences controlling the expression of a selection cassette, as both GS KO-GB24 and ADCC+-GB24 pools generated with these vectors show an increase in productivity in relation to the pools transfected with the vector containing the HSVmin promoter WT sequence.

### Example 8: Use of a Post-Translational Modification Cassette

A transposon vector comprising a post-translation modification cassette, such as the ST6Gal1 gene, and a selection marker, such as the neomycin resistance gene, as illustrated in FIG. 9B, was co-transfected with transposase mRNA into GS KO cells via electroporation. 48 h after transfection, cells were placed in selection media (CD OptiCHO+™ containing 4 mM glutamine and neomycin) for 8 days. To develop pools expressing both ST6Gal1 and trastuzumab (Ttz), the ST6Gal1 pools generated previously were co-transfected with the transposase mRNA and a transposon vector expressing Ttz and comprising the GB24 promoter controlling the expression of the selection cassette glutamine synthetase. Stably ST6Gal1 and Ttz expressing pools were generated in glutamine free media, which was initiated 48 h after transfection. Selection was completed and pools were expanded to E125 flasks when cell density had increased on two consecutive counts and was above 0.5×10⁶ and viability had increased between two consecutive counts and was above 70%. Non-transfected cells were used as a control for the selection process. mRNA level of ST6Gal1 in stably expressing pools was assessed in the recovered cells using RT-qPCR. RNA was extracted from 1×10⁶ cells using the PureLink™ RNA Mini kit (Invitrogen™) following manufacturer's instructions. cDNA was prepared using 1 μg of RNA and Superscript IV and random hexamers. For qPCR analysis, 10 ng of cDNA was used as input together with gene specific primers. The qPCR was run on Agilent Mx3000™ using PowerUp™SYBR™ Green master mix (Applied Biosystems). GAPDH was used as a reference gene and cDNA from non-transfected GS KO cells was used as a control. Fold changes were calculated using the AACT method.

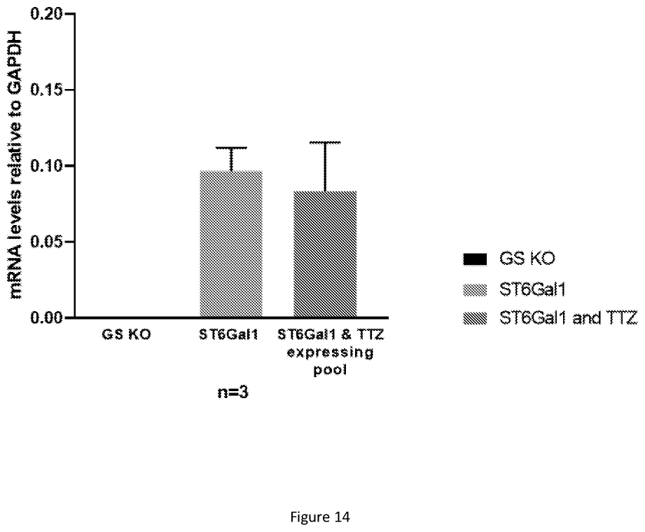

FIG. 14 shows the ST6Gal1 mRNA levels in stably expressing pools detected by RT-qPCR. The results show that ST6Gal1 mRNA was not detected in the non-transfected control sample GS KO and that pools expressing either ST6Gal1 or both ST6Gal1 and Ttz had an increased level of ST6Gal1 mRNA. The results indicate that the use of a post-translational modification cassette, as illustrated in FIG. 9 or together with the expression of Ttz, successfully led to the production of ST6Gal1 in transfected GS KO cells.

After confirming the stably expressing pools and the mRNA levels, a 14-day fed-batch experiment was conducted using pools expressing only Trastuzumab (Ttz) and pools co-expressing Ttz and ST6Gal1. Ttz antibody, present in the supernatants of these pools harvested on day 14, was purified using Protein A HP SpinTrap™ column (Cytiva™). The eluted Ttz was then used for N-glycan analysis.

N-Glycan analysis was performed on the LabChip® GXII Touch™ (PerkinElmer™) with the GXII Glycan Release and Labeling Kit (PerkinElmer™) Glycan LabChip® Reagent Kit (PerkinElmer™), and High-Resolution Chip (PerkinElmer) as per the manufacturer's instructions. The data was analysed with LabChip® GX Reviewer software and aligned to glycan standards that were previously run.

Electropherograms were compared and aligned for the glycan standards (see FIG. 19(A)) as well as for the transfected pools, to detect and identify differences in the N-glycan profile. The cells in which Ttz and ST6Gal1 were co-expressed, mono-sialylated and di-sialylated peaks can be detected, i.e., F(6)A2G(4)2S(6)1 (peak 1) and F(6)A2G2S(6)2 (peak 2), respectively, as shown in FIG. 19 (B). In contrast, in the cells expressing only Ttz, the sialylated peaks were not detected.

N-Glycan analysis data shows that in cells co-transfected with a post-translation modification cassette, comprising the ST6Gal1 gene and a vector expressing Ttz and comprising the GB24 promoter controlling the expression of the selection cassette glutamine synthetase, a shift towards the mono- and di-sialylated structures was observed, compared to the Ttz-expressing cells where the glycans are non-sialylated, indicating that the produced ST6Gal1 protein was capable of modifying an antibody expressed by the polynucleotide of the present disclosure.

### Example 9: Expression of Single-Chain Proteins

A transposon vector expressing the GFP protein in a single expression cassette and comprising the GB14 promoter controlling the expression of the selection cassette glutamine synthetase was co-transfected with transposase mRNA into GS KO cells via electroporation. Four pools were generated in total. 48 hours post transfection, all pools were resuspended in selection media (CD OptiCHO™ glutamine free media). Following recovery, cells were subcultured twice a week for two weeks in CD OptiCHO™ selection media. GFP expression was analysed using the Sony SH800S™ cell sorter and the manufacturer's recommendations, 1×10⁶ cells were taken from each pool and 10,000 events/pools were analysed. Non-transfected GS KO cells were used as control for the FACS analysis. Live cells were isolated from the total number of events by gating for granularity and pulse diameter using SSC-A/FSC-A. Subsequently, single cells were analysed by gating for pulse height and diameter using FSC-H/FSC-A.

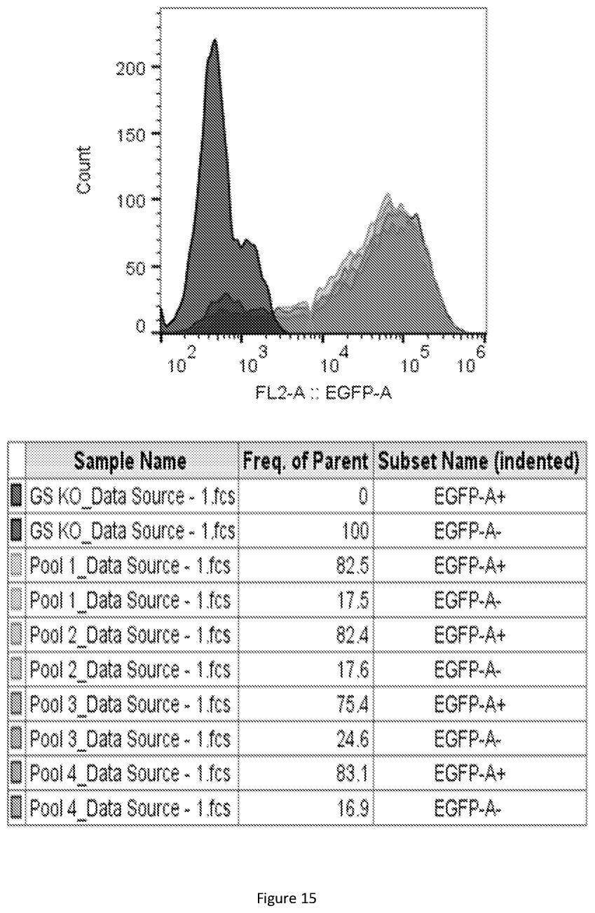

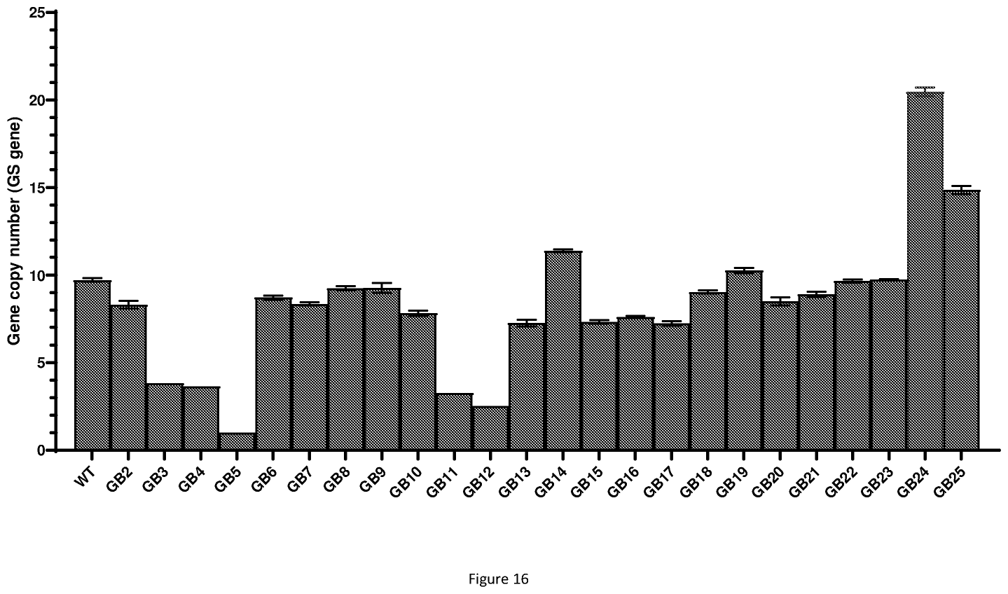

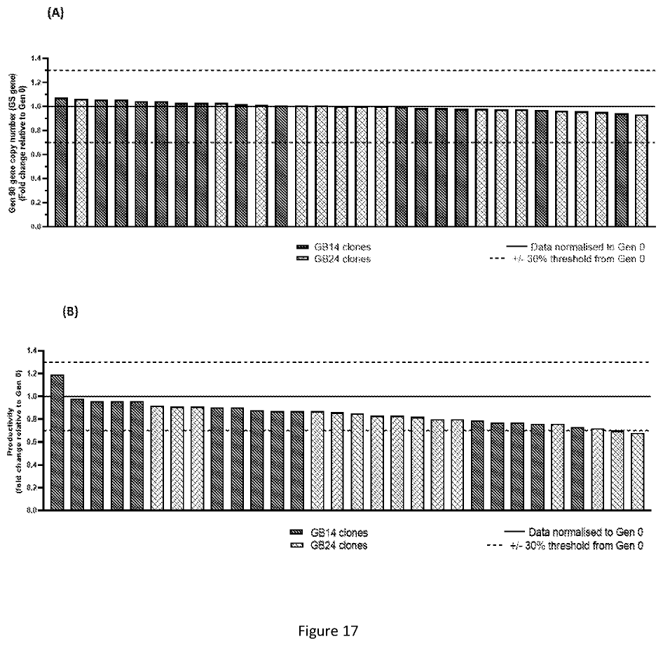

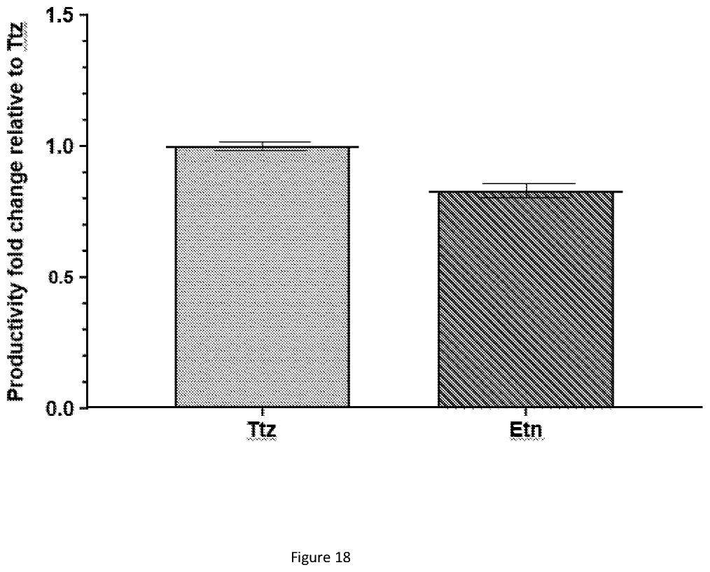

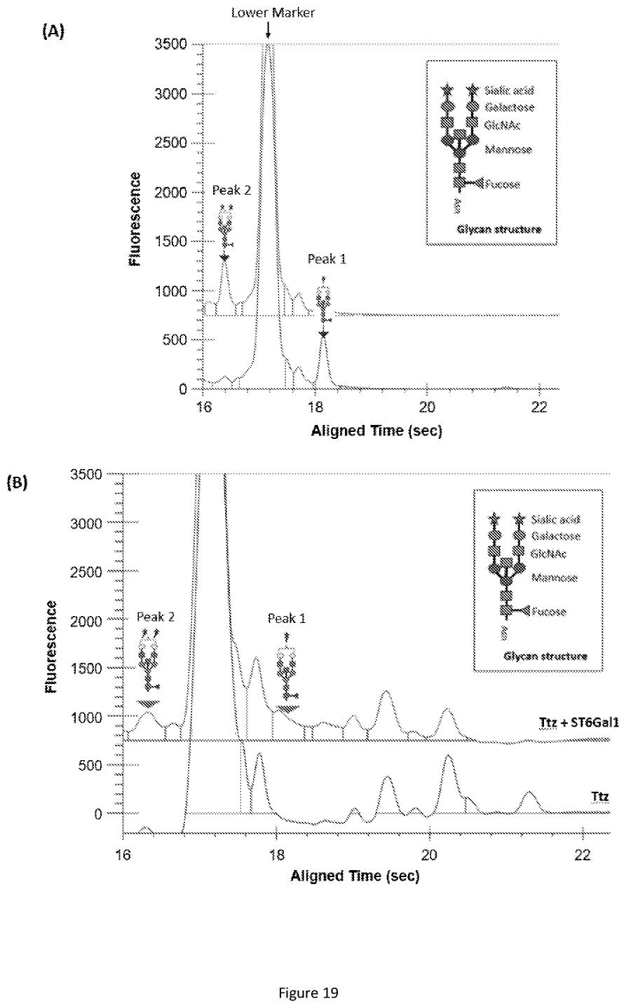

FIG. 15 shows the GFP expression analysis in stably expressing GS KO pools. The results show that at least 75% of cells from the pools transfected using the GFP expressing vectors were positive for GFP expression. In contrast, no GFP expression was measured in the non-transfected GS KO cell line. These results indicate that GFP was successfully expressed from the transposon vector comprising the GFP expression cassette and the selection cassette under the control of the GB14 promoter.

Transposon vectors comprising the polynucleotides of the present disclosure including either light and heavy chain genes encoding Ttz antibody as the expression cassette and glutamine synthetase as the selection cassette, or a gene encoding Etn fusion protein and glutamine synthetase as selection cassette were co-transfected with transposase mRNA into GS KO cells via electroporation. The methods for generating stably expressing pools and for conducting the 14-day fed-batch overgrowth experiment are described in Example 7.

FIG. 18 shows the productivity profile of Ttz and Etn during the 14-day fed-batch overgrowth culture. The results confirm that expression vectors comprising GB24 promoter sequences controlling the expression of a selection cassette can be used to express different classes of protein biotherapeutics, such as the Ttz antibody and the fusion protein Etn.

TABLE 3
Copy number variation (CNV),
recovery day and nucleotide sequence of
the HSVmin promoters tested

| | | Re- | |
|---|---|---|---|
| Sequence | | covery | |
| ID | CNV | Day | Sequence |
| WT | 9.7 | 5 | CAGCTGCTTCATCCCCGTGGCCCGTT |
| (SEQ ID | | | GCTCGCGTTTGCTGGCGGTGTCCCC |
| NO: 1) | | | GGAAGAAATATATTTGCATGTCTTT |
| | | | AGTTCTATGATGACACAAACCCCGC |
| | | | CCAGCGTCTTGTCATTGGCGAATTC |
| | | | GAACACGCAGATGCAGTCGGGGCG |
| | | | GCGCGGTCCCAGGTCCACTTCGCAT |
| | | | ATTAAGGTGACGCGTGTGGCCTCGA |
| | | | ACACCGAGCGACCCTGCAG |
| GB2 | 8.3 | 5 | CTATGATGACACAAACCCCGCCCAG |
| (SEQ ID | | | CGTCTTGTCATTGGCGAATTCGAAC |
| NO: 2) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB3 | 3.8 | 18 | TAATACAAACCCCGCCCGAATTCGA |
| (SEQ ID | | | ACACGCAGATGCAGTCGGGGCGGC |
| NO: 3) | | | GCGGTCCCAGGTCCACTTCGCATAT |
| | | | TAAGGTGACGCGTGTGGCCTCGAAC |
| | | | ACCGAGCTAG |
| GB4 | 3.7 | 18 | TAATCGTCTTGTCATTGGCGAATTCG |
| (SEQ ID | | | AACACGCAGATGCAGTCGGGGCGG |
| NO: 4) | | | CGCGGTCCCAGGTCCACTTCGCATA |
| | | | TTAAGGTGACGCGTGTGGCCTCGAA |
| | | | CACCGAGCTAG |
| GB5 | 1.0 | 22 | TAATGAACACGCAGATGCAGTCGGG |
| (SEQ ID | | | GCGGCGCGGTCCCAGGTCCACTTCG |
| NO: 5) | | | CATATTAAGGTGACGCGTGTGGCCT |
| | | | CGAACACCGAGCTAG |
| GB6 | 8.7 | 5 | CTATGATGACACAAACCCCGCCCAG |
| (SEQ ID | | | CGTCTTGTCATTGGCGAATTCGAAC |
| NO: 6) | | | ACGCAGATGCAGTCGGGGCGGCGC |
| | | | GGTCCGAGGTCCACTTCGCATATTA |
| | | | AGGTGCCGGATCCGGCCTCGAACAC |
| | | | CGAGCGACCCTGCAGC |
| GB7 | 8.4 | 5 | CTATGATGACACAAACCCCGCCCAG |
| (SEQ ID | | | CGTCTTGTCATTGGCGAATTCGAAC |
| NO: 7) | | | CCGGATCCGGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB8 | 9.2 | 5 | CTATGATGACACAAACCCCGCCCAG |
| (SEQ ID | | | CGTCTTGTCATTGGCCCGGATCCGG |
| NO: 8) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB9 | 9.3 | 5 | CTATGATGACACAAACCCCGCCCAG |
| (SEQ ID | | | CGTCTTGTCATTGGCGCCGGATCCG |
| NO: 9) | | | GCGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB10 | 7.8 | 5 | CTATGATGACACAAACCCCGCCCAG |
| (SEQ ID | | | CGTCTTGTCATTGGCGAATTCGAAC |
| NO: 10) | | | ACGCAGATGCAGTCGGGGCGGCGC |
| | | | GGTCCCGGATCCGGTTCGCATATTA |
| | | | AGGTGACGCGTGTGGCCTCGAACAC |
| | | | CGAGCGACCCTGCAGC |
| GB11 | 3.3 | 14 | TAATACAAACCCCGCCCGAATTCGA |
| (SEQ ID | | | ACACGCAGATGCAGTCGGGGCGGC |
| NO: 11) | | | GCGGTCCCAGGTCCACTTCGCATAT |
| | | | TAAGGTGACGCGTGTGGCCTCGAAC |
| | | | ACCGAGCGACCCTGCAGCGACCCGC |
| | | | TTAGCTAG |
| GB12 | 2.5 | 14 | TAATCGTCTTGTCATTGGCGAATTCG |
| (SEQ ID | | | AACACGCAGATGCAGTCGGGGCGG |
| NO: 12) | | | CGCGGTCCCAGGTCCACTTCGCATA |
| | | | TTAAGGTGACGCGTGTGGCCTCGAA |
| | | | CACCGAGCGACCCTGCAGCGACCCG |
| | | | CTTAGCTAG |
| GB13 | 7.3 | 5 | CTATGATGACACAAACCCCGCCCAG |
| (SEQ ID | | | CGTCTTGTCATTGGCGAATTCGAAC |
| NO: 13) | | | ACGCAGATGCAGTCGGGGCGGCGC |
| | | | GGTCCGAGGTCCACTTCGCATATTA |
| | | | AGGTGACGCGTGTGCCGGATCCGGC |
| | | | CGAGCGACCCTGCAGC |
| GB14 | 11.4 | 6 | CTATGATGACACAAACCCCGCCCAG |
| (SEQ ID | | | CGTCTTGTCATTGGCGAATTCGAAC |
| NO: 14) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGCCGGATCCGGCAGC |
| GB15 | 7.3 | 5 | CCCGGATCCGGCAAACCCCGCCCAG |
| (SEQ ID | | | CGTCTTGTCATTGGCGAATTCGAAC |
| NO: 15) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB16 | 7.6 | 5 | CTATGATGACACAAACCCCGCCCAG |
| (SEQ ID | | | CGTCTTGTCATTGGCGAATTCGAAC |
| NO: 16) | | | ACGCAGATGCAGTCGGGGCGGCGCC |
| | | | GGATCCGGTCCACTTCGCATATTAA |
| | | | GGTGACGCGTGTGGCCTCGAACACC |
| | | | GAGCGACCCTGCAGC |
| GB17 | 7.2 | 5 | CTATGCCGGATCCGGCCCCGCCCAG |
| (SEQ ID | | | CGTCTTGTCATTGGCGAATTCGAAC |
| NO: 17) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB18 | 9.0 | 5 | CTATGATGACACAAACCCCGCCCAG |
| (SEQ ID | | | CCGGATCCGGTTGGCGAATTCGAAC |
| NO: 18) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB19 | 10.2 | 7 | CTATGATGACACAAACCCCGCCCAG |
| (SEQ ID | | | CGTCTTGTCATTGGCGAATTCGAAC |
| NO: 19) | | | ACGCAGATGCAGTCCCGGATCCGGG |
| | | | GTCCGAGGTCCACTTCGCATATTAA |
| | | | GGTGACGCGTGTGGCCTCGAACACC |
| | | | GAGCGACCCTGCAGC |
| GB20 | 8.5 | 5 | CTATGATGACACAAACCCCGCCCAG |
| (SEQ ID | | | CGTCTTGTCATCCGGATCCGGGAAC |
| NO: 20) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB21 | 8.9 | 5 | CTATGATGACACAAACCCCGCCCAG |
| (SEQ ID | | | CGTCTTGTCATTGGCGAATTCGAAC |
| NO: 21) | | | ACGCAGATGCACCGGATCCGGCGCG |
| | | | GTCCGAGGTCCACTTCGCATATTAA |
| | | | GGTGACGCGTGTGGCCTCGAACACC |
| | | | GAGCGACCCTGCAGC |
| GB22 | 9.6 | 5 | CTATGATGACACAAACCCCGCCCAG |
| (SEQ ID | | | CGTCTTGTCATTGGCGAATTCGAAC |
| NO: 22) | | | ACGCAGATGCAGTCGGGGCGGCGC |
| | | | GGTCCGAGGTCCACTTCGCATATTA |
| | | | CCGGATCCGGGTGTGGCCTCGAACA |
| | | | CCGAGCGACCCTGCAGC |
| GB23 | 9.7 | 5 | CTATGATGACACAAACCCCGCCCAG |
| (SEQ ID | | | CGTCTTGTCATTGGCGAATTCGAAC |
| NO: 23) | | | ACGCAGATGCAGTCGGGGCGGCGC |
| | | | GGTCCGAGGTCCACTTCCCGGATCC |
| | | | GGTGACGCGTGTGGCCTCGAACACC |
| | | | GAGCGACCCTGCAGC |
| GB24 | 20.5 | 11 | CTATGATGACCGGATCCGGGCCCAG |
| (SEQ ID | | | CGTCTTGTCATTGGCGAATTCGAAC |
| NO: 24) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |
| GB25 | 14.9 | 10 | CTATGATGACACAAACCGGATCCGG |
| (SEQ ID | | | CGTCTTGTCATTGGCGAATTCGAAC |
| NO: 25) | | | ACGCAGATGCAGTCGGGGCGGCGC |
| | | | GGTCCGAGGTCCACTTCGCATATTA |
| | | | AGGTGACGCGTGTGGCCTCGAACAC |
| | | | CGAGCGACCCTGCAGC |

TABLE 4
Summary of the findings

| | | | Productivity (Fold |
|---|---|---|---|
| Sequence | | Recovery | change relative to |
| ID | CNV | Day | WT) |
| WT | 9.7 | 5.0 | 1.0 |
| GB14 | 11.4 | 6.0 | 1.2 |
| GB24 | 20.5 | 11.0 | 2.1 |

1. A polynucleotide comprising:
a selection cassette, wherein the selection cassette comprises:
an HSVMin promoter sequence and a selection marker sequence, wherein the HSVMin promoter sequence is from 120 to 160 nucleotides long and comprises;
(a) SEQ ID NO: 14 or SEQ ID NO: 24,
(b) sequence complementary to SEQ ID NO: 14 or SEQ ID NO: 24,
(c) a sequence comprising at least 80% sequence identity to SEQ ID NO: 14 or SEQ ID NO: 24 or
a sequence comprising at least 80% sequence identity to a sequence complementary to SEQ ID NO: 14 or SEQ ID NO: 24 over a span of at least 100 nucleotides.

2. The polynucleotide of claim 1, further comprising one or more expression cassettes.

3. The polynucleotide of claim 2, wherein the polynucleotide comprises a cargo region comprising the selection cassette and the one or more expression cassettes.

4. The polynucleotide of claim 2, wherein the one or more expression cassettes are oriented in a first direction and the selection cassette is oriented in a second direction, wherein the first direction and the second direction are opposite directions.

5. The polynucleotide of claim 2, wherein the one or more expression cassettes comprise a first expression cassette and a second expression cassette, wherein the first expression cassette and the second expression cassette are oriented in a first direction.

6. (canceled)

7. The polynucleotide of claim 5, wherein the selection cassette is located between the first expression cassette and the second expression cassette.

8. The polynucleotide of claim 1, wherein the selection marker sequence is a glutamine synthetase sequence, and wherein the glutamine synthetase sequence codes for a glutamine synthetase protein.

9. The polynucleotide of claim 5, further comprising a third expression cassette.

10. The polynucleotide of claim 3, wherein the polynucleotide is a vector.

11. The polynucleotide of claim 10, wherein the vector is a transposon vector, a retroviral vector or a lentiviral vector.

12.-13. (canceled)

14. The polynucleotide of claim 10, wherein the vector is a transposon vector and the cargo region is flanked by a sequence or a pair of sequences that are recognized by a Tc1/mariner transposase.

15. The polynucleotide of claim 14, wherein the Tc1/mariner transposase protein is a Sleeping Beauty transposase.

16.-19. (canceled)

20. The polynucleotide of claim 5, wherein either:
(a) the first expression cassette comprises a nucleotide sequence that codes for an antibody heavy chain and the second expression cassette comprises a nucleotide sequence that codes for an antibody light chain; or
(b) the first expression cassette comprises a nucleotide sequence that codes for an antibody light chain and the second expression cassette comprises a nucleotide sequence that codes for an antibody heavy chain.

21.-28. (canceled)

29. The polynucleotide of claim 8, wherein the glutamine synthetase sequence comprises or is complementary to SEQ ID NO: 26 and the HSVMin promoter sequence comprises or is complementary to SEQ ID NO: 14 or SEQ ID NO: 24.

30.-42. (canceled)

43. A genetic delivery system comprising the transposon vector of claim 11 and either a transposase protein, a DNA plasmid, or an mRNA, wherein the DNA plasmid or the mRNA encode the transposase protein.

44. A method for integrating an exogenous nucleotide sequence into a nucleotide sequence in a host cell, said method comprising; introducing into the host cell the polynucleotide of claim 1.

45.-52. (canceled)

53. A method for integrating an exogenous nucleotide sequence into a nucleotide sequence in a host cell, said method comprising; introducing into the host cell the polynucleotide of claim 1, under conditions that allow for random integration.

54. A cell comprising the polynucleotide of claim 1.

55.-60. (canceled)

61. A method of generating a biologic material, a therapeutic protein or a non-protein therapeutic biologic, said method comprising culturing the cell of claim 54.

62.-68. (canceled)

69. A kit for generating and modifying a protein, wherein the kit comprises the polynucleotide of claim 1, wherein the polynucleotide is a first polynucleotide, and the kit further comprises a second polynucleotide,
wherein the first polynucleotide comprises at least one expression cassette that comprises a nucleotide sequence that codes for a polypeptide, and
wherein the second polynucleotide comprises a post-translational modification cassette, and the post-translational modification cassette codes for an amino acid sequence of a protein that is capable of a post-translational modification of the polypeptide coded by the first polynucleotide.

70.-85. (canceled)
